(19) United States
(12) Patent Application Publication
(10) Pub. No.: US 2009/0017105 A1
(43) Pub. Date: Jan. 15, 2009

(54) PROLIPOSOMAL AND LIPOSOMAL COMPOSITIONS OF POORLY WATER SOLUBLE DRUGS

(76) Inventors: Dhiraj Khattar, Ghaziabad (IN); Mukesh Kumar, Ghaziabad (IN); Rama Mukherjee, Ghaziabad (IN); Anand C. Burman, Sahibabad (IN); Minakshi Garg, Ghaziabad (IN); Manu Jaggi, Ghaziabad (IN); Anu T. Singh, Ghaziabad (IN); Anshumali Awasthi, Ghaziabad (IN)

Correspondence Address: SCHWEGMAN, LUNDBERG & WOESSNER, P.A., P.O. BOX 2938, MINNEAPOLIS, MN, 55402, US

(21) Appl. No.: 12/045,958

(22) Filed: Mar. 11, 2008

(30) Foreign Application Priority Data
Mar. 19, 2007 (IN) 590/DEL/2007

(51) Int. Cl. A61K 9/127; A61P 35/00

(52) U.S. Cl. 424/450

(57) ABSTRACT

Concentrates or proliposomal compositions of poorly water-soluble drugs and compounds, comprising of one or more membrane forming lipids, a membrane stabilizing agent, in a suitable vehicle, and optionally containing a Polyethylene Glycol (PEG)-coupled phospholipid or a mixture thereof and further, optionally containing pharmaceutically acceptable excipients such as antioxidants, buffering agents, acidifying agents etc. are provided, which have superior long term stability. The concentrates of proliposomal compositions instantly form liposomes of the said poorly water-soluble drugs and compounds on rapid injection to a diluting fluid, the liposomal composition so obtained, characterized by a physical stability more than 24 hours, ≧95% drug encapsulation and having a particle size diameter of less than 100 nm. The liposomal compositions so obtained can further be directly administered to patients in need of treatment of the poorly water-soluble drugs and compounds.

### FIELD OF THE INVENTION

The invention relates to concentrates or proliposomal compositions of poorly water-soluble drugs and compounds, comprising of one or more membrane forming lipids, selected from a saturated and/or an unsaturated phospholipid; a membrane stabilizing agent, selected from a sterol compound; in a suitable vehicle, selected from a water-miscible solvent or mixtures thereof; and the composition optionally containing one or more of a Polyethylene Glycol (PEG)-coupled phospholipid and further, optionally containing pharmaceutically acceptable excipients such as antioxidants, buffering agents, acidifying agents etc.

The invention further relates to use of the concentrates or proliposomal compositions for preparation of liposomal compositions of the poorly water-soluble drugs and compounds in particle size diameter of less than 100 nm, instantly at the bedside of patients, which is not only simple, convenient, cost-effective and safe for administration to patients in need thereof but also exhibit improved stability and higher drug retention.

### BACKGROUND OF THE INVENTION

There is an ever-increasing interest and demand for a delivery system of drugs and compounds, especially poorly water-soluble drugs and compounds, which are not only stable, have optimum drug loading, are preferably in a nanoparticulate form and which, moreover, are simple, convenient and safe for administration to patients in need thereof.

Amongst such delivery systems, proliposomal and liposomal compositions have held and continue to hold an important position in research endeavours world over since the early 1960s, when it was first observed that lipid vesicles could encapsulate certain chemical compounds. Since then and particularly in the last few years, the research endeavours have gathered great momentum with the objective of encapsulating-life saving drugs and compounds in lipid vesicles as well as with the objective of not only improving or enhancing the therapeutic efficacy of the said drugs but also their safety, toxicity, pharmacokinetic, pharmacodynamic, bioavailability, targeted action, and other related properties or profiles through administration of such drug-encapsulated lipid-vesicles. This has culminated in commercialization of a few technologies and subsequent introduction to the market place of a few liposomal drug delivery systems, which offer great advantages over conventional delivery systems comprising such drugs and compounds.

Sears in U.S. Pat. No. 4,426,330 and U.S. Pat. No. 4,534,899 was among the first to disclose a synthetic phospholipid and its use in preparation of liposomal compositions of poorly water-soluble drugs, such as Paclitaxel and Hexamethylmelamine, as well as water-insoluble fragrance oils for cosmetic uses.

However, apart from the advancement of art the method of Sears in U.S. Pat. No. 4,426,330 and U.S. Pat. No. 4,534,899 has achieved, there is very little knowledge about the effectiveness of the method in delivery of poorly water-soluble drugs such as Paclitaxel into the blood stream.

Bally et al. in U.S. Pat. No. 5,077,056 disclose a method for encapsulation of ionisable antineoplastic agents in liposomes to an extent as high as 99% using transmembrane potentials as well as disclose use of such transmembrane potentials to reduce the rate of release of ionisable drugs from liposomes. The method involves establishing a pH gradient across a liposome bilayer such that the ionisable drug to be encapsulated within a liposome is uncharged in the external buffer and charged within the aqueous interior, allowing the drug to readily cross the liposomal bilayer in the neutral form and be trapped within the aqueous interior of the liposome due to conversion of the charged form.

However, the main disadvantage or limitation of the method disclosed by Bally et al. in U.S. Pat. No. 5,077,056 is the leakage of the drug from actively loaded liposomes, following the loss of proton gradient.

Barenholz et al. in U.S. Pat. No. 4,797,285 and U.S. Pat. No. 4,898,735 disclose a liposomal composition of the anthracycline glycoside, Doxorubicin, present in a mole percent of about 2.5 in the composition, further comprising of 20-50 mole percent of cholesterol; 10-40 mole percent of a negatively charged phospholipid; a water-soluble tihydroxamic chelating agent, namely ferrioxamine in a concentration of about 50 μM; and α-Tocopherol in a concentration of at least 0.2 mole percent, the latter two components acting as free-radical scavengers.

However, the drug entrapment in the liposomes disclosed by Barenholz et al. in U.S. Pat. No. 4,797,285 and U.S. Pat. No. 4,898,735, at the best is not more than 85-90%, with a lot left to be desired.

Ogawa et al. in U.S. Pat. No. 5,094,854 disclose liposomal compositions, utilizing membrane phospholipids, of which the acyl groups are saturated and having a phase transition temperature of 40° C. and 45° C., wherein a drug-containing solution having an osmotic pressure 1.2 to 2.5 times higher than of the body fluid of warm-blooded animals is entrapped.

However, from the enabling experimental details given by Ogawa et al. in U.S. Pat. No. 5,094,854, with respect to the rate of release of the anticancer drug, Cisplatin (CDDP), it could be seen that the rate of release of the drug at 39° C. was hardly anything, whereas at 42° C. the rate of release varies from 30 to 95%.

Woodle et al. in U.S. Pat. No. 5,013,556 disclose liposomal compositions of drugs, consisting of between 1-20 mole percent of an amphipathic lipid derivatized with a polyalkylether, which are reported to have significant circulation time in the blood stream.

It would appear that the enhanced circulation time in the blood stream observed, is probably because of utilization of phospholipids derivatized with polyethylene glycol (PEG), a phenomenon well known prior to the disclosure of Woodle et al. in U.S. Pat. No. 5,013,556.

Huang et al. in WO 92/02208 disclose a lyophilized liposomal composition of the anthracycline glycoside, Doxorubicin, reported to be stable against Doxorubicin breakdown on long term storage. The liposomal composition is characterized by the presence of neutral phospholipids, cholesterol, a negatively charged lipid, and a bulking agent, with a drug:lipid ratio of between 5-10% by weight and a Doxorubicin concentration of less than 10 mg/ml.

However, the potency of Doxorubicin in the liposomal composition disclosed by Huang et al. in WO 92/02208 was found to drop by 10-15% in two weeks, suggesting that the lyophilized composition ought to be utilized as quickly after its preparation for reconstitution with a suitable fluid for administration to patients.

Rahman et al. in U.S. Pat. No. 5,424,073 and U.S. Pat. No. 5,648,090 disclose a liposomal-encapsulated composition of the anticancer drug, Paclitaxel or Taxol, which was reported to have advantages over the other known compositions of Paclitaxel or Taxol in that the said liposomal delivery system helped in avoidance of the solubility problem of the drug as well as anaphylactoid reactions and cardiotoxicity; led to improved stability and therapeutic efficacy of the drug; rendered administration of the drug as a bolus or short infusion rather than extended (24 hour) infusion; aided modulation of multidrug resistance in cancer cells etc.

The liposomal composition disclosed by Rahman et al. in U.S. Pat. No. 5,424,073 and U.S. Pat. No. 5,648,090 essentially comprised of one, wherein Taxol is encapsulated in a lipid vehicle made up of negative, positive, and neutral liposomes, with a concentration of about 9.5 to 10 mole percent of Taxol. Such Taxol-encapsulated liposomes are reported to be prepared by first mixing together a solution of Taxol in a suitable non-polar or polar solvent with a solution of the lipid-forming material in a solvent having low polarity, followed by removal of solvents from the mixture to afford a thin, dry film of the lipid and the drug, to which was added saline solution to form the liposomes. Examples 1 to 4, described therein claim that the encapsulation efficiency of Taxol in the said liposomes was more than 95%. It is further claimed that aliquots of such liposomes were stable for four days and for one month at room and refrigeration temperatures respectively.

Furthermore, Rahman et al. in U.S. Pat. No. 5,424,073 and U.S. Pat. No. 5,648,090 claim that Taxol liposomes prepared in the abovementioned manner, with the only difference of substituting saline solution with a 7% trehalose-saline solution for re-suspension of the liposomes were stable at −20° C. and −80° C. for one month and five months respectively, with intermittent thawing of the liposomes, leading to an inference that Taxol liposomes with trehalose as excipient can be an effective means of storing the frozen liposomes, that can be further effectively used for clinical and therapeutic applications, after thawing of such frozen liposomes.

The foremost limitation of the liposomal composition disclosed by Rahman et al. in U.S. Pat. No. 5,424,073 and U.S. Pat. No. 5,648,090 lies in their method of preparation thereof in that it is well known that liposomes in general have very little survival rate in saline solutions and break down very rapidly. This, in fact has been the finding of Fang et al., as reported in Chem. Pharm. Bull., 1997, 45(9), 1504-1509, which states that liposomes with cholesterol underwent hydrolysis after incubation with normal saline. Secondly, while such liposomes show some stability in presence of trehalose, a diglucose sugar, however, it should not be forgotten that whatever stability achieved could not be possible without freezing the liposomes to temperatures of between −20° C. and −80° C., which needless to mention, increase their cost of manufacture and thereby, restrict their commercial application.

Staubinger et al. in U.S. Pat. No. 5,415,869 disclose liposomal compositions of taxanes, including Taxol, which comprises encapsulation of the said taxane in a lipid vehicle consisting of a mixture of one or more negatively charged phospholipids and one or more zwitterion i.e. uncharged phospholipids. Staubinger et al., further specify that the ratio of the negatively charged phospholipids to the zwitterion phospholipids that can be employed are in the range of 1:9 to 7:3, with the concentration of the taxane present in the liposomal composition being in an amount of 1.5 to 8.0 mole percent.

Staubinger et al. in U.S. Pat. No. 5,415,869 further claim that the liposomal compositions of taxanes thus produced are in the form of particles having a size of 0.025 to 10 microns and the composition is substantially free of any taxane crystal formation.

Furthermore, Staubinger et al. in U.S. Pat. No. 5,415,869 claim that by virtue of utilization of the combination of the negatively charged and the zwitterion phospholipids in the specified ratio helps not only in prevention of aggregation or fusion of the liposomes but also in prevention of crystal formation, which render safe intravenous administration of the composition as well as render circulation of the drug for longer periods of time.

While, no doubt, the liposomal compositions disclosed by Staubinger et al. in U.S. Pat. No. 5,415,869 constitute a substantial advance in the art related to liposomal technology, however, prima facie, the technology suffers from an inherent disadvantage or limitation in that the loading of the drug i.e. taxanes in the object liposomal compositions is in the range of 1.5 to 8.0 mole percent only, which is abysmally low for any drug. Further, the molar ratio of the taxane:lipid employed is approximately 1:33, again indicative of the poor drug loading. Secondly, contrary to the claims, there is no suggestion in the Specification that the liposomes have extended circulation lives. Finally, the subject liposomal compositions after their preparation are lyophilized, which calls for special manufacturing facilities, which is expensive and tends to be the privy of only select manufacturers. In short, the liposomal compositions disclosed by Staubinger et al., does not elicit any commercial application, thereby rendering such methods and compositions as of academic interest only.

Durr et al. in U.S. Pat. No. 5,670,536 disclose a liposomal composition of the anticancer drug, Docetaxel or a taxoid derived from Docetaxel, comprising at least one unsaturated phospholipid and at least one negatively charged phospholipid, subject to that the said unsaturated and negatively charged phospholipids are different from one another.

Durr et al. in U.S. Pat. No. 5,670,536 further recite a method for preparation of the object liposomal compositions of Docetaxel or a taxoid derived from Docetaxel, the method essentially comprising of dissolving the drug and the respective lipids in a non-toxic organic solvent, preferably an alcohol, followed by evaporation of the solvent under an inert atmosphere and under reduced pressure to afford a solvent-free gel or syrupy paste, to which is further added water or a 0.9% aqueous sodium chloride solution and homogenized to obtain a fine dispersion. To the dispersion is added a cytoprotective agent, intended for prevention of crystallization of the active drug and/or for adjustment of the tonicity of the solution and finally, the dispersion is subjected to sterile filtration and either lyophilized or frozen to provide the object liposomal compositions of Docetaxel or a taxoid derived from Docetaxel.

Durr et al. in U.S. Pat. No. 5,670,536 mention that the liposomal compositions thus obtained remain clear for more than eight weeks at 20° C. and have a particle diameter of between 47 to 71 nm. It is further claimed that the compositions have the advantage of incorporating the active principle or drug, without any crystallization or precipitation occurring.

At best, the disclosure of Durr et al. in U.S. Pat. No. 5,670,536 can be considered as an extension of the work reported by Staubinger et al. in U.S. Pat. No. 5,415,869 as far as prevention of crystallization or precipitation of the active principle or drug is concerned, the only difference being that the former replaces the zwitterion phospholipid with an unsaturated phospholipid. While, the disclosure of Durr et al. talks about better stability and higher level of the active principle or drug, however, at least on the first count, the reported stability appear to be inferior to that disclosed by Staubinger et al. Further, the disclosure of Durr et al. is silent about the amount of drug encapsulated in the lipid vehicle. Furthermore, the method of Durr et al., like that Staubinger et al. also involves a step of lyophilization or freezing of the liposomes, which, as mentioned hereinbefore, calls for special manufacturing facilities, which is expensive and tends to be the privy of only a select manufacturers. Finally, the liposomal composition of Durr et al. may have very little survival rate in saline solutions and could break down very rapidly, as has been the finding of Fang et al., as reported in Chem. Pharm. Bull., 1997, 45(9), 1504-1509.

Leigh et al. in U.S. Pat. No. 5,004,611 and U.S. Pat. No. 5,141,674 disclose a proliposomal composition of biologically active compounds, comprising at least one membrane lipid; at least one non-toxic water-miscible organic liquid, which is a solvent for the lipid; and up to 40% by weight of water, with the proportion by weight of the lipid to the organic liquid being from 40:1 to 1:20. Suitable membrane lipids disclosed are natural lecithins, such as soy lecithin and egg yolk lecithin as well as synthetic lecithins, such as di-palmitoyl phosphatidyl choline or others such as glycolipids, long chain dialkyl dimethyl ammonium compounds, di-tallow ammonium compounds etc. These proliposomal compositions are reported to be progenitors of liposomes and accordingly Leigh et al. also disclose the utility of such proliposomal compositions for preparation of liposomal compositions of the said biologically active compounds comprising the method of mixing the proliposomal compositions with water. It is further stated that the liposomes so formed have diameters in the range of 0.1 to 2.5 microns and contain at least 2 ml of entrapped aqueous fluid per gram of the lipid and are further characterized by the presence of detectable quantities of the water-miscible organic liquid in the aqueous dispersion. Furthermore, it is stated that the liposomal compositions so formed are advantageously provided as aerosol formulations, comprising the said liposomal compositions in a volatile liquid propellant.

While, Leigh et al. in U.S. Pat. No. 5,004,611 and U.S. Pat. No. 5,141,674 teach the utility of the proliposomal compositions of biologically active compounds in preparation of liposomal compositions of the said biologically active compounds by mixing the former with water, however, from Table-I described therein, it would be abundantly evident that the method results in rather poor entrapment of the said biologically active compounds, with the entrapment efficiency ranging from as low as 22% to as high as 45% only, which is abysmally low by any standard and does not merit any commercial application.

Hager et al. in U.S. Pat. No. 5,556,637 and U.S. Pat. No. 5,741,517 in another variant, provide a water-containing liposome system for pharmaceutically active substances, containing at least one phospholipidic charge carrier, preferably a negatively charged phospholipid, in addition to at least one uncharged phospholipid, which moreover, is claimed to have high stability and does not tend to from sediments.

While, the pharmaceutically active substances disclosed by Hager et al. in U.S. Pat. No. 5,556,637 and U.S. Pat. No. 5,741,517 comprise Doxorubicin hydrochloride, Pentamidine, a Pentamidine salt, Rosemarinic acid, a salt of Rosemarinic acid, Quinoline yellow and Dextran sulphate, however, from the enabling description of the liposomal systems of the abovementioned substances, as evident from the Examples given therein, it would be evident that the encapsulation efficiency or capacity of such systems are not quite satisfactory, for e.g. the encapsulation of Doxorubicin hydrochloride, reported being only 78%, whereas in the case of Quinoline yellow the liposome-bound active principle constitutes only 1.38 mg/ml, while the non-liposomal-bound active principle is found to constitute about 3.2 mg/ml.

Fisher et al. in U.S. Pat. No. 6,132,763 disclose liposomal compositions for delivery of drugs and contrast agents for Magnetic Resonance (MR) imaging, wherein external surface of the liposomes are covalently linked to a Poly Ethylene Glycol (PEG) moiety. Such liposomes having PEG moieties covalently bound to phospholipids on the external surface are reported to extend the circulation life-time of the liposomes without disrupting the lipid bi-layer. The covalently bonded PEG-liposomes are further prepared by treatment of the liposomes with a reactive derivative of PEG, such as 2,2,2-trifluoroethanesulfonyl (tresyl) monomethoxy PEG.

The method disclosed by Fisher et al. in U.S. Pat. No. 6,132,763 for preparation of the PEGylated liposomes is highly sensitive and requires great skill and dexterity in their preparation for achieving the desired results.

In a departure from the abovementioned methods, Mayhew et al. in U.S. Pat. No. 5,939,567 and U.S. Pat. No. 6,118,011 disclose preparation of a taxane derivative, wherein a hydrophobic moiety is attached to either the 2′- or 7-positions or both the positions of the taxane skeleton, with the result that such modified taxane derivatives are found to generally stabilize the association of the said derivative with a lipid, including a liposomal lipid. Also provided are compositions of such modified taxanes containing a lipid carrier in a pharmaceutically acceptable medium. The hydrophobic organic moieties include saturated or unsaturated, aliphatic or branched fatty acids, polyols, sphingolipids etc.

While, from the data provided by Mayhew et al. in U.S. Pat. No. 5,939,567 and U.S. Pat. No. 6,118,011, it would be apparent that introduction of a hydrophobic moiety into the taxane skeleton vastly improves the percentage of drug encapsulated in the liposomes, e.g. about 90% entrapment of 7-caproyl Paclitaxel, and about 70% entrapment of 2′-caproyl Paclitaxel, as compared to about 20% entrapment of Paclitaxel, however, even 90% of drug entrapment is not satisfactory or adequate from a commercial point of view, since other liposomal compositions of Paclitaxel, without any hydrophobic moiety at the 2′- or 7-positions achieve a drug entrapment of >95%.

Kim et al. in U.S. Pat. No. 5,720,976 disclose thermosensitive liposomal compositions, comprising drug-entrapped liposomes coated with copolymer of N-isopropylacrylamide, octadecylacrylate, or acrylic acid, which release the drug at variable temperatures by control of the acrylic acid content in the copolymer.

The disadvantage with the liposomal compositions disclosed by Kim et al. in U.S. Pat. No. 5,720,976 is related to the use of acrylic acid based copolymers, the safety of such copolymers in pharmaceutical preparations being questionable.

Needham et al. in U.S. Pat. No. 6,200,598 and U.S. Pat. No. 6,726,925 B1 disclose thermosensitive liposomal compositions of an active agent, comprising a gel-phase lipid bilayer membrane having a phase transition temperature of between 39° C. to 45° C. and one or more lysolipids, characterized by having an acyl group, wherein the amount of an surface active agent contained in the gel-phase bilayer membrane is sufficient to increase the percentage release of the active agent at the phase transition temperature of the bilayer compared to that would occur in the absence of the surface active agent. Further, the presence of the surface active agent is reported to stabilize rather than destabilize the membrane, particularly prior to the melting of the lipid bilayer.

Needham et al. in U.S. Pat. No. 6,200,598 and U.S. Pat. No. 6,726,925 B1 claim that the liposomes so formed have a size from about 50 nm to 500 nm in diameter. Further, from the release profile of 6-carboxyfluorescein (CF) disclosed therein it could be seen that incorporation of as little as 10 mole % of the lysolipid, Monopalmitoylphosphatidylcholine (MPCC) as surface active agent results in nearly four fold increase in the release of CF, compared to those where MPCC is absent. However, in terms of entrapment of the active agent, within the liposomes, a lot more would be desired, if one takes the example of entrapment of Doxorubicin, wherein the entrapment of the drug is not more than 80%.

Staubinger et al. in U.S. Pat. No. 6,348,215 B1 provide a method for stabilization of a taxane, especially Taxol present in a liposome system by exposing the said taxane-containing liposome to a molecule, which improves the physical stability of the taxane. Of the molecules, which are reported stabilize the taxanes is a glycerol-water mixture, wherein the glycerol present in the mixture acts as the molecule or others such as CH₃, acetic acid and acetic anhydride. From the results summarized in Tables 1 and 2 therein, it could be seen that when different proportions of glycerol-water are used, generally Paclitaxel exhibits stability up to 6 hours.

While, the disclosure of Staubinger et al. in U.S. Pat. No. 6,348,215 B1 is generally concerned about improvement of the entrapped taxane in the liposomal composition, however, it is silent about the degree of entrapment of the drug in the liposomes.

Webb et al. in US Patent Application No. 2005/0118249 A1 disclose liposomal compositions of biologically active agents, comprising at least one vesicle forming lipid and at least one aggregation preventing component, characterized in that the composition contains less than 20 mole percent of cholesterol and that the intraliposomal aqueous medium has an osmolarity of 500 mOsm/kg or less.

The method disclosed by Fisher et al. in U.S. Pat. No. 6,132,763 is highly sensitive and successful preparation of the object liposomes largely depend on obtaining the right pH gradient, which calls for great skill and dexterity in their preparation.

Tardi et al. in US Application No. 2005/0118250 A1 also disclose liposomal compositions of biologically active agents, comprising of at least one vesicle forming lipid; at least 1 mole percent of a negatively charged lipid comprising a zwitterions moiety, which is an aggregation preventing agent and which also contains less than 20 mole percent of cholesterol.

The limitation of the method disclosed by Tardi et al. in US Application No. 2005/0118250 A1 is that the liposomes prepared are stored either as a lyophilized powder or frozen and further require the presence of cryoprotectants, which collectively increase the cost of manufacture of such liposomes, thereby rendering them as not particularly attractive, commercially.

Boni et al. in US Application No. 2003/0224039 A1 disclose a method for entrapment of a bioactive agent in a liposome or lipid complex comprising infusion of an lipid-ethanol solution into an aqueous or ethanolic solution of the bioactive agent, at a temperature below the phase transition of at least one of the lipid components of the lipid-ethanol solution and preferably above the surface of the solution.

It is, however, not very clear from the disclosure of Boni et al. in US Application No. 2003/0224039 A1 the degree of entrapment of the bioactive agents in the liposomes, following the method described therein.

MacLachlan et al. in US Application No. 2004/0142025 A1 disclose processes and apparatus for preparation of lipid vesicles that optionally contain a therapeutic agent, the process typically comprising first providing an aqueous solution in a first reservoir, which is in fluid communication with an organic lipid solution, optionally containing a therapeutic agent in a second reservoir and mixing the aqueous solution with the organic lipid solution, wherein the organic lipid solution undergoes a continuous stepwise dilution to produce a liposome.

The method disclosed by MacLachlan et al. in US Application No. 2004/0142025 A1 is highly sensitive and complex and requires critical supervision for preparation of liposomes having the desired characteristics.

Hoarau et al. in US Application No. 2005/0214378 A1 disclose stealth lipid nanocapsules, essentially consisting of a lipid core, which is liquid or semi-liquid; an outer lipid envelope comprising at least one hydrophobic surfactant and at least one lipophilic surfactant, which are lipid in nature; and at least one amphiphilic derivative of polyethyleneglycol (PEG), the molar mass of the PEG component of which is greater than or equal to 2000 gm/mol, with the PEGylated amphiphilic derivative conferring the stealth aspect on the nanocapsules, in turn allowing incorporation and transport of molecules and active principles transported in dissolved or dispersed form.

The method for preparation of the stealth lipid nanocapsules, as disclosed by Hoarau et al. in US Application No. 2005/0214378 A1, appear to be highly sensitive and tedious and therefore, would call for critical supervision of the manufacturing process as well would require great skill and dexterity in their manufacture

Kozubek et al. in WO 2005/072776 A2 disclose liposomal formulations of antineoplastic agents, incorporating in the formulations semi-synthetic polyhydroxyl derivatives of alkylphenols, which result in high encapsulation efficiency of the active substance to the tune of >90%.

However, the method disclosed by Kozubek et al. in WO 2005/072776 A2 for preparation of the object liposomal formulations involve a two-stage lyophilization and/or freezing process, which not only increases the cost of manufacture but also requires capital investment for installation of expensive lyophilizers, which is the privy of select manufacturers.

Bhamidipati in US Application No. 2006/0034908 A1 disclose a method for large scale manufacture of liposomal compositions comprising addition of a lipid fraction and an active principle in t-butanol to an aqueous solution and mixing the mixture at a temperature of between 20° C. to 40° C. to form the bulk liposomal preparation, which can be further processed by size fractionation or reduction, removal of the solvent, sterilization by membrane filtration, freeze drying or other methods.

It is not clear as to what is the speciality of the method disclosed by Bhamidipati in US Application No. 2006/0034908 A1 compared to those known and practiced in the art for bulk liposomal preparations.

Edgerly-Plug et al. in U.S. Pat. No. 6,596,305 B1 disclose a method for preparation of a population of liposomes, having a desired mean particle size, comprising the steps of forming a mixture of vesicle-forming lipids in a single phase solvent system containing a water-miscible organic solvent and water, the controlling of the mean particle size of the liposomes being achieved by adjustment of the initial concentration of the solvent in the said solvent system.

Here again, it is not clear as to what is the speciality of the method disclosed by Edgerly-Plug et al. in U.S. Pat. No. 6,596,305 B1 compared to those known and practiced in the art for bulk liposomal preparations.

From the foregoing, it would be abundantly evident that while the abovementioned disclosures have to great extent made advances to the liposomal technology, however, most, if not all of them suffer from one or more of the following limitations, which render them as not having an universal application for preparation of liposomal drug delivery systems for biologically active compounds, and more specially poorly water-soluble drugs and compounds. Some of the limitations are:
i) crystallization or precipitation of the active principles from the liposomal compositions;
ii) inadequate storage stability, compounded by leakage of the active principle from the liposomes over a period of time;
iii) poor and inconsistent entrapment or encapsulation of the active principles in the lipid layer, varying from as low as 20% to as high as 95%;
iv) very high drug:lipid ratio, in a few cases as high as 1:33;
v) lyophilization of the liposomal compositions in majority of the instances, which not only increases the cost of manufacture but also necessitates capital investment in installation of a lyophilizer, which is the privy of only a select manufacturers;
vi) freezing of the liposomal compositions at temperatures as low as from −20° C. and −80° C. for storage, which also significantly increases the cost of manufacture as well as cost of transportation or shipment and storage of the said liposomal compositions;
vii) utilization of cryoprotectants in variable proportions in the compositions, which also increase the cost of manufacture;
viii) utilization of acrylic acid based copolymers, the safety of such copolymers in many preparations, especially pharmaceutical preparations being questionable;
ix) utilization of highly sensitive methods, especially for preparation of the PEGylated liposomes, which require great skill and dexterity in their preparation for achieving the desired results;
x) employment of and dependency on highly critical and sensitive parameters and controls, such as intraliposomal osmolarity, pH gradient, phase transition temperature, reactors and apparatus etc. for release of the active principle as well as stability of the liposomal compositions, which again calls for critical supervision, and great skill and dexterity in their preparation;
   employment of fluids, especially saline solutions for reconstitution of the liposomes, which have a tendency to degrade the liposomes rapidly, etc.

Further, most of the abovementioned disclosures primarily discuss the degree of entrapment or encapsulation of active principles in the lipid layer as well as their stability per se, with all of the disclosures either silent or not having made any attempt for providing an active principle in its maximum potency on administration to a patient in need thereof. It need not be over emphasized that most, if not all of the prior art liposomal compositions have been reported to have a stability of only a few weeks, if not a few days and the time such compositions are manufactured, stored, shipped and reconstituted for administration to a patient, some, if not significant loss in potency of the entrapped or encapsulated active principle would be inevitable, with the result that the patient does not get the full benefit of receiving a more potent drug for treatment.

To the present inventors, this has been a grave omission from the research endeavours of the peers and no matter whatever advances have been made for preparation of the liposomes, equal importance or advances ought to have been made for providing the active principle at its optimum potency at the time of reconstitution and subsequent administration to a patient in need thereof.

A need, therefore, exists for a liposomal composition for a wide host of drugs, especially poorly water-soluble drugs and compounds, which are free or substantially free of the limitations associated with the prior art compositions, and which, moreover, can be manufactured in a cost effective manner and furthermore, can be reconstituted very conveniently, preferably at the bedside of patients, thereby ensuring that the patient gets the benefit of the maximum potency of the administered drug.

The present invention is a step forward in this direction and provides a concentrate or proliposomal composition of poorly water-soluble drugs and compounds, which can be manufactured in a simple, convenient and inexpensive manner, and which, moreover, has high storage stability. The present invention further provides a method of preparation of liposomal compositions of poorly water-soluble drugs and compounds utilizing the concentrate or proliposomal compositions of such poorly water-soluble drugs or compounds, which is simple, convenient and most importantly, unlike the prior art methods, is prepared and obtained on reconstitution with a suitable diluting fluid at the bedside of patients and, which, in turn can be immediately-administered to patients in need thereof at its optimum potency. The liposomal compositions of poorly-water soluble drugs and compounds of the present invention are characterised by a vastly improved or superior stability and a drug loading as high as 95% as or >95%.

### OBJECTS OF THE INVENTION

An object of the present invention, of utmost importance and significance, is to provide concentrates or proliposomal compositions of poorly water-soluble drugs and compounds of high storage stability, which in turn can be utilized for instant preparation of liposomal compositions of such poorly water-soluble drugs and compounds on reconstitution with a suitable diluting fluid at the bedside of the patient and thereafter can be instantly administered to a patient in need of the poorly water-soluble drugs and compounds at its optimum potency.

Another object of the present invention is to provide concentrates or proliposomal compositions of poorly water-soluble drugs and compounds, which are free of the limitations, associated with the prior art compositions.

Yet another object of the present invention is to provide liposomal compositions of poorly water-soluble drugs and compounds, which are free of the limitations, associated with the prior art compositions.

Still another object of the present invention is to provide liposomal compositions of poorly water-soluble drugs and compounds, possessing high stability and a drug loading as high as 95% or >95%.

A further object of the present invention is to provide a process for preparation of concentrates or proliposomal compositions of poorly water-soluble drugs and compounds, which is simple, convenient and cost-effective.

Another object of the present invention is to provide a process for preparation of concentrates or proliposomal compositions of poorly water-soluble drugs and compounds, which does not require employment of and dependency on highly critical and sensitive parameters and which, moreover, does not call for critical supervision, and great skill and dexterity in their preparation.

Yet another object of the present invention is to provide a process for preparation of liposomal compositions of poorly water-soluble drugs and compounds, which is simple, convenient and cost-effective.

Still another object of the present invention is to provide a process for preparation of liposomal compositions of poorly water-soluble drugs and compounds, which does not require employment of and dependency on highly critical and sensitive parameters and which, moreover, does not call for critical supervision, and great skill and dexterity in their preparation

A further object of the present invention is to provide a process for preparation of liposomal compositions of poorly water-soluble drugs and compounds from a concentrate or proliposomal compositions comprising the said poorly water-soluble drugs and compounds, instantly on reconstitution with a suitable diluting fluid at the bedside of the patient.

Another object of the present invention is to provide a process for preparation of liposomal compositions of poorly water-soluble drugs and compounds, which provides the liposomes, having consistent particle size.

Yet another object of the present invention is to provide a method for treatment of pathological conditions, which the poorly water-soluble drugs and compounds are capable of, comprising administration of liposomal compositions of such poorly water-soluble drugs and compounds, which are prepared instantly on reconstitution of the concentrates or proliposomal compositions of such poorly water-soluble drugs or compounds with a suitable diluting fluid at the bedside of the patient in need of the treatment.

Still another object of the present invention is to provide a method for treatment of pathological conditions, which the poorly water-soluble drugs and compounds are capable of, comprising administration of liposomal compositions of such poorly water-soluble drugs and compounds at their optimum potency, which in turn are prepared instantly on reconstitution of the concentrates or proliposomal compositions of such poorly water-soluble drugs or compounds with a suitable diluting fluid at the bedside of the patient in need of the treatment.

Another object of the present invention is to provide concentrates or proliposomal compositions of poorly water-soluble drugs and compounds in a suitable kit, convenient for preparation of Liposomal compositions of such poorly water-soluble drugs and compounds on reconstitution with a suitable diluting fluid.

### DESCRIPTION OF THE DRAWINGS AND FIGURES

FIG. 1: Comparison of the in vivo Antitumour Activity of a Liposomal Composition of Docetaxel, as per the Present Invention and that of the Conventional Composition of Docetaxel, Taxotere® in B16.F10 Xenograft.

FIG. 2: Comparison of the Body Weights of C57BL/6 Mice treated with a Liposomal Composition of Docetaxel, as per the Present Invention and that of the Conventional Composition of Docetaxel, Taxotere®.

FIG. 3: Comparison of Dose-Kinetics for Tubulin Polymerization obtained with a Liposomal Composition of Docetaxel, as per the Present Invention and that of the Conventional Composition of Docetaxel, Taxotere® in Ovarian Cancer Cells.

FIG. 4: Comparison of Time-Kinetics for Tubular Polymerization obtained with a Liposomal Composition of Docetaxel, as per the Present Invention and that of the Conventional Composition of Docetaxel, Taxotere® in PA1 Cell Line at 1 μM.

FIG. 5: Dose-Kinetics for Tubulin Polymerization obtained with a Liposomal Composition of Docetaxel, as per the Present Invention and that of the Conventional Composition of Docetaxel, Taxotere® in Ovarian Cancer Cells.

FIG. 6: Time-Kinetics for Tubulin Polymerization obtained with a Liposomal Composition of Docetaxel, as per the Present Invention and that of the Conventional Composition of Docetaxel, Taxotere® in Ovarian Cancer Cells.

### SUMMARY OF THE INVENTION

In their endeavours to meet the objectives, in the first place, the present inventors have found that concentrates or proliposomal compositions of poorly water-soluble drugs, comprising of:
a) a poorly water-soluble drug or compound as the active principle;
b) a membrane forming lipid, comprising of one or more of a saturated phospholipid or an unsaturated phospholipid or mixtures thereof;
c) a membrane stabilizing agent, selected from a sterol compound;
d) a vehicle for the lipids, selected from a water-miscible organic solvent or mixtures thereof; and
e) optionally containing one or more of a Polyethylene Glycol (PEG)-coupled phospholipid; and further
f) optionally containing pharmaceutically excipients, such as antioxidants, buffering agents, or acidifying agents;
   with the active principle present in the concentrate or composition in mole percent of between 9 to 14; the membrane forming saturated phospholipid present in the concentrate or composition in mole percent of between 40 to 50; the membrane forming unsaturated phospholipid present in the concentrate or composition in mole percent of between 15 to 20; the membrane stabilizing sterol compound present in the concentrate or composition in mole percent of between 25 to 35, and optionally an antioxidant present in the concentrate or composition in mole percent of between 0.20 to 1.0; and further optionally a Polyethylene Glycol (PEG)-coupled phospholipid present in the concentrate or composition in mole percent of between 2 to 5, could be prepared in a simple, convenient, and cost-effective manner, which, moreover, is easily amenable to large scale manufacture. The concentrates or compositions may further optionally contain a buffering agent or an acidifying agent, in quantities essential to adjust the pH of the solution and/or stabilization of the composition.

The concentrates or proliposomal compositions of poorly water-soluble drugs thus obtained, do not require either to be lyophilized or frozen at cryogenic temperatures for storage and as such, the concentrates or proliposomal compositions of the present invention are found to possess enhanced stability at ambient or refrigeration temperatures. This has significant advantages in that it brings down the cost of manufacture considerably.

For instance, a concentrate or proliposomal composition of the anticancer drug, Docetaxel in a mole percent of between 9 to 11, comprising of Hydrogenated soy phosphatidyl choline (HSPC) as the saturated membrane forming lipid in a mole percent of between 43 to 45, Egg Phosphatidyl Glycerol (EPG) as the unsaturated membrane forming lipid in a mole percent of between 16 to 18, and cholesterol as the membrane stabilizing agent in a mole percent of between 25 to 27, in about 1 ml of ethanol as the vehicle was found to be stable for at least 6 months at 25±2° C. and at 60±5% RH, with drop in assay of Docetaxel from the initial value 9.5-mg/ml to 9.1 mg/ml only and further found to equally stable for at least 6 months at 2-8° C., with drop in assay of Docetaxel from the initial value 9.5 mg/ml to 9.1 mg/ml only. The compositions remained clear, without any observable sedimentation for the six-month period it was observed.

Similarly, a concentrate or proliposomal composition of the anticancer drug, Docetaxel in a mole percent of between 9 to 11, comprising of Hydrogenated soy phosphatidyl choline (HSPC) as the saturated membrane forming lipid in a mole percent of between 43 to 45, Egg Phosphatidyl Glycerol (EPG) as the unsaturated membrane forming lipid in a mole percent of between 16 to 18, and cholesterol as the membrane stabilizing agent in a mole percent of between 25 to 27, and α-tocopherol as the antioxidant in a mole percent of 1.0, in about 1 ml of ethanol as the vehicle was found to be stable for at least 6 months at 25±2° C. and at 60±5% RH, with drop in assay of Docetaxel from the initial value 9.2 mg/ml to 8.7 mg/ml only and further found to equally stable for at least 6 months at 2-8° C., with drop in assay of Docetaxel from the initial value 9.2 mg/ml to 8.8 mg/ml only. The compositions remained clear, without any observable sedimentation for the six-month period it was observed.

Further, a concentrate or proliposomal composition of the anticancer drug, Docetaxel in a mole percent of between 9 to 11, comprising of Hydrogenated soy phosphatidyl choline (HSPC) as the saturated membrane forming lipid in a mole percent of between 43 to 45, Egg Phosphatidyl Glycerol (EPG) as the unsaturated membrane forming lipid in a mole percent of between 16 to 18, and cholesterol as the membrane stabilizing agent in a mole percent of between 25 to 27, in a about 1 ml mixture containing ethanol and propylene glycol in a ratio of 9:1 as the vehicle was found to be stable for at least 3 months at 25±2° C. and at 60±5% RH, with no drop in assay of Docetaxel from the initial value 8.8 mg/ml to 8.9 mg/ml only and further found to be equally stable for at least 3 months at 2-8° C., with again no drop in assay of Docetaxel from the initial value 8.8 mg/ml to 8.8 mg/ml only. The compositions remained clear, without any observable sedimentation for the three-month period it was observed.

Furthermore, a concentrate or proliposomal composition of the anticancer drug, Docetaxel in a mole percent of between 9 to 11, comprising of Hydrogenated soy phosphatidyl choline (HSPC) as the saturated membrane forming lipid in a mole percent of between 43 to 45, Egg Phosphatidyl Glycerol (EPG) as the unsaturated membrane forming lipid in a mole percent of between 16 to 18, and cholesterol as the membrane stabilizing agent in a mole percent of between 25 to 27, a Polyethylene Glycol (PEG)-coupled phospholipid (MPEG 2000-DSPE) in a mole percent of between 2 to 3, in about 1 ml of ethanol as the vehicle was found to be stable for at least 6 months at 25±2° C. and at 60±5% RH, with drop in assay of Docetaxel from the initial value 9.1 mg/ml to 8.7 mg/ml only and further found to be equally stable for at least 6 months at 2-8° C., with again drop in assay of Docetaxel from the initial value 9.1 mg/ml to 8.7 mg/ml only. The compositions remained clear, without any observable sedimentation for the six-month period it was observed.

The abovementioned results on stability of the concentrate or proliposomal composition of Docetaxel are summarized in Table-I, given at a later part of this specification.

The other advantage the concentrates or proliposomal compositions of the present invention offers is that virtue of their enhanced stability, even at ambient or refrigeration temperatures, the said concentrates or compositions could be stored for prolonged period of time, without significant loss in potency of the active principle and also could be transported under such storage conditions in a more convenient manner, which moreover, significantly brings down the cost of transportation as well storage in warehouses.

The concentrates or proliposomal compositions of the poorly water-soluble drugs or compounds as active principles, in turn can be manufactured by a simple and convenient method comprising mixing together the respective proportions of the active principle, the membrane forming lipids, the membrane stabilizing agent and optionally the Polyethylene Glycol (PEG)-coupled phospholipid and/or the pharmaceutically acceptable excipients in the vehicle, which normally is one or more of a water-miscible organic solvent to obtain a solution, followed by sterile filtration into containers for storage. The method does not call for adherence to any critical parameter or operation and thereby does away with any critical supervision and moreover, does not require any skill or dexterity on the part of the operator for manufacture of the object concentrates or proliposomal compositions.

In other endeavours to meet the objectives, the present inventors have found that the concentrates or proliposomal compositions of poorly water-soluble drugs or compounds, as discussed and obtained hereinbefore, could be conveniently utilized for formation, preparation, or manufacture of liposomal compositions of poorly water-soluble drugs or compounds instantly at the bedside of patients in need of treatment or administration of the said poorly water-soluble drugs or compounds, through a simple operation of injection of the said concentrate or proliposomal compositions into a suitable diluting fluid for administration, which can be carried out safely by a practicing doctor or other qualified medical or para-medical supervisors or staff.

The liposomes were formed instantly on injection of the concentrates or proliposomal compositions into the diluting fluid. While, there could be some variation in the mean particle size diameter of the liposomes so formed, however, it is an aspect of the present invention that liposomes of consistent particle size diameter of less than 100 nm, can be obtained, produced, or manufactured in the diluting fluid for reconstitution by injection of the concentrates or proliposomal compositions, and through syringes with hypodermic needles having a gauge of between 18 G to 30 G, at a rate of about 0.10 ml/second to about 1.5 ml/second. Further, the degree of entrapment or encapsulation of the poorly water-soluble drugs or compounds in the liposomes was found to be very high and in most instances it was found to be about 95% or more than 95%.

The liposomes thus obtained, produced, or manufactured in the diluting fluid for reconstitution, apart from having the advantage of being obtained, produced, or manufactured in consistent particle size diameter of less than 100 nm in most instances, are found to possess significantly higher physical stability in the reconstitution medium, for instance a physical stability of not less than 4 hours, and in many instances ≧24 hours, depending of the nature of the poorly water-soluble drug or compound entrapped or encapsulated in the liposomes.

For instance, a liposomal composition of the anticancer drug, Docetaxel, prepared by injection of a concentrate or proliposomal composition of the same in a mole percent of between 9 to 11, comprising of Hydrogenated soy phosphatidyl choline (HSPC) as the saturated membrane forming lipid in a mole percent of between 44 to 46, Egg Phosphatidyl Glycerol (EPG) as the unsaturated membrane forming lipid in a mole percent of between 16-18, and cholesterol as the membrane stabilizing agent in a mole percent of between 26 to 27, into a 5% Dextrose solution as the diluting fluid, through syringes with hypodermic needles having a gauge of between 18 G to 30 G, at a rate of about 0.10 ml/second to about 1.5 ml/second was found to have a particle size diameter of about 95 nm and having a physical stability of more than 12 hours, with no crystallization or precipitation of the drug from the reconstituted media. Further, the entrapment or encapsulation of the drug in the liposomes was found to be greater than 95%.

Further, since, by virtue of the enhanced storage stability of the concentrates or proliposomal compositions as well by virtue of the instant preparation or manufacture of the respective liposomal compositions at the bedside of patients, a great benefit is conferred upon the patients receiving administration of the said liposomal compositions in that they get the drug administered in its optimum potency, bettering their chances to an early recovery from the pathological disorders they are suffering from. Furthermore, by virtue of the instant preparation or manufacture of the respective liposomal compositions at the bedside of patients, there is no requirement for a dedicated manufacturing facility, with special emphasis on sterile manufacturing, which becomes a cost-effective feature of the present invention.

From the foregoing, it would be abundantly evident that both the concentrates or proliposomal compositions and the liposomal compositions, obtained from the former offer greater advantages over the respective prior art compositions in terms of:
i) higher storage and physical stability of both the compositions;
ii) greater than 95% entrapment or encapsulation of the active principle in the liposomes;
iii) manufacture of liposomes of the active principles consistently in particle size diameter of less than 100 nm;
iv) simple, convenient, and cost-effective or inexpensive process for preparation of both the compositions;
v) the preparation or manufacture of both the compositions not requiring any critical supervision as well as any great skill or dexterity from the personnel preparing or manufacturing the same; and
vi) providing the patients in need of administration of the liposomal compositions the benefit of receiving the active principles at its optimum potency,
   thereby meeting most, if not all of the objectives set forth.

In further endeavours to meet the objectives, the present inventors have found it convenient to provide the concentrates or proliposomal compositions of poorly water-soluble drugs and compounds in a suitable sterile container as a kit along with a container comprising of an appropriate or suitable diluting fluid, wherein the former can be conveniently injected into the latter for reconstitution and formation of the liposomes, as per the details mentioned hereinbefore and subsequent administration of the reconstituted liposomes to patients in need of treatment.

It is found advantageous to provide in the kit the concentrates or proliposomal compositions of poorly water-soluble drugs and compounds in sterile glass vials or vials made up of other non-toxic materials, along with a container comprising of an appropriate or suitable diluting fluid, the material of construction of the said container again can be glass or other non-toxic materials. The concentrate or proliposomal composition can be withdrawn from its container by a syringe, having needle specifications, as mentioned hereinbefore and then injected into the container, holding the diluting fluid, at a rate as specified hereinbefore to obtain the liposomal composition of the poorly water-soluble drugs and compounds, ready for administration to patients in need thereof.

It is also found advantageous to provide in the kit, a pre-filled sterile syringe containing the concentrates or proliposomal compositions of poorly water-soluble drugs and compounds, along with a suitable hypodermic needle having the specified gauge of 18 G to 30 G, as mentioned hereinbefore, further along with a container comprising of an appropriate or suitable diluting fluid, the material of construction of the said container again can be glass or other non-toxic materials. The concentrates or proliposomal composition contained in the pre-filled syringe can then be injected with the aid of the needles provided, directly into the container holding the diluting fluid, at a rate as specified hereinbefore, to obtain the liposomal composition of the poorly water-soluble drugs and compounds, ready for administration to patients in need thereof.

### DETAILED DESCRIPTION OF THE INVENTION

The present invention is detailed as hereinunder:

### Concentrates or Proliposomal Compositions of Poorly Water-Soluble Drugs and Compounds of the Present Invention

As mentioned hereinbefore, the concentrates or proliposomal compositions of poorly water-soluble drugs, as per the present invention comprises of:
a) a poorly water-soluble drug or compound as the active principle;
b) a membrane forming lipid, comprising of one or more of a saturated phospholipid or an unsaturated phospholipid or mixtures thereof;
c) a membrane stabilizing agent, selected from a sterol compound;
d) a vehicle for the lipids, selected from a water-miscible organic solvent or mixtures thereof; and
e) optionally containing one or more of a Polyethylene Glycol (PEG)-coupled phospholipid; and further
f) optionally containing pharmaceutically excipients, such as antioxidants, buffering agents, or acidifying agents.

Poorly water-soluble drugs or compounds are those having water solubility of less than 10 mg/ml. Examples of such poorly water-soluble drugs or compounds include, but are not limited to, anticancer agents, anti-inflammatory agents, anti-fungal agents, antiemetics, antihypertensive agents, sex hormones, steroids, antibiotics, immunomodulators, anaesthetics etc. Typical examples of anticancer agents that can be utilized in the concentrates or proliposomal compositions of the present invention include Paclitaxel, Docetaxel, and other related taxane derivatives; Irinotecan, Topotecan, SN-38 and other related Camptothecin derivatives; Doxorubicin, Daunomycin, and related Anthracycline Glycosides; Cisplatin; Oxaliplatin; 5-Fluorouracil, Mitomycin; Methotrexate; Etoposide; Betulinic acid and its derivatives; and Wedelolactone and its derivatives. Typical examples of anti-inflammatory agents that can be utilized in the concentrates or proliposomal compositions of the present invention include Indomethacin, Ibuprofen, Ketoprofen, Flubiprofen, Piroxicam, Tenoxicam, and Naproxen. Typical examples of anti-fungal agents that can be utilized in the concentrates or proliposomal compositions of the present invention include Ketoconazole, and Amphotericin B. Typical examples of sex hormones that that can be utilized in the concentrates or proliposomal compositions of the present invention include Testosterone, Estrogen, Progesterone, and Estradiol. Typical examples of steroids that that can be utilized in the concentrates or proliposomal compositions of the present invention include Dexamethasone, Prednisolone, Fulvestrant, Exemestane and Triamcinolone. Typical examples of antihypertensive agents that that can be utilized in the concentrates or proliposomal compositions of the present invention include Captopril, Ramipril, Terazosin, Minoxidil, and Parazosin. Typical examples of antiemetics that that can be utilized in the concentrates or proliposomal compositions of the present invention include Ondansetron and Granisetron. Typical examples of antibiotics that that can be utilized in the concentrates or proliposomal compositions of the present invention include Metronidazole, and Fusidic acid. Typical examples of immunomodulators that that can be utilized in the concentrates or proliposomal compositions of the present invention include Cyclosporine; and Biphenyl dimethyl dicarboxylic acid. Typical examples of anaesthetics that that can be utilized in the concentrates or proliposomal compositions of the present invention include Propofol, Alfaxalone, and Hexobarbital

With regard to anticancer agents in particular, various Betulinic acid derivatives, such as those designated as MJ-1098, DRF-4012 and DRF-4015 having the following structures (I), (II), and (III), which in turn are disclosed in U.S. Pat. No. 6,403,816 and our PCT Application No. WO 2006/085334 A2, also qualify as poorly water-soluble drugs and compounds and can be utilized in the concentrates or proliposomal compositions of the present invention.

The poorly water-soluble drugs and compounds can be employed in mole percent of between 9 to 14 in the concentrates or proliposomal compositions, preferably in mole percent of between 9 to 11.

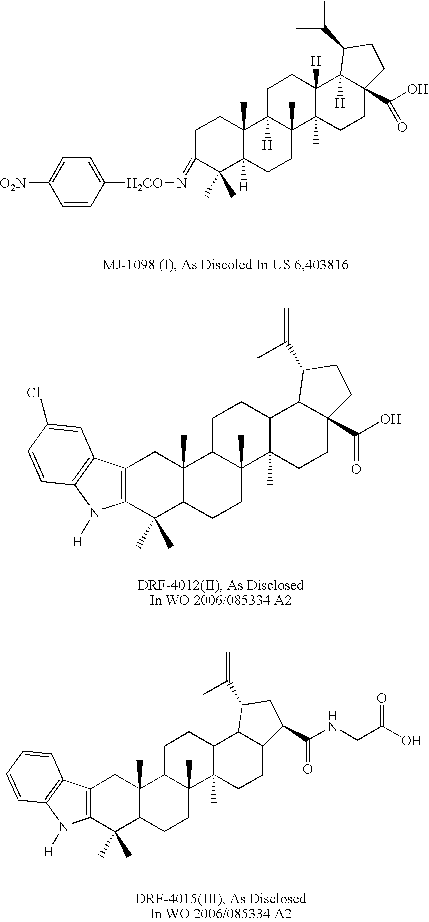

The membrane forming lipids that can be employed in the concentrates or proliposomal compositions can be one of an unsaturated phospholipid, a saturated phospholipid or a mixture thereof.

The unsaturated phospholipids that can be employed in the concentrates or proliposomal compositions of the present invention are selected from Lecithin, Phosphatidylcholine (PC), Phosphatidyl ethanolamine (PE), Lysolecithin, Lysophosphatidyl ethanolamine, Dilaurylphosphatidyl choline (DLPC), Dioleoyl phosphatidyl choline (DOPC), Sphingomyelin, Brain Sphingomyelin, Cerebrosides, Egg Phosphatidyl glycerol (EPG), Soya phosphatidyl glycerol (SPG), Phosphatidyl inositol (PI), Phosphatidic acid (PA), Phosphatidyl serine (PS), Dilauroyl phosphatidyl glycerol (DLPG), Cardiolipins and mixtures thereof.

The unsaturated phospholipids can be employed in the range of between 15 to 20 mole percent of the total concentrates or proliposomal compositions. The unsaturated phospholipids can be a zwitterionic or anionic in nature. A preferred unsaturated phospholipid is Egg Phosphatidyl glycerol (EPG).

The saturated phospholipids that can be employed in the concentrates or proliposomal compositions of the present invention are selected from the group consisting of Hydrogenated soya phosphatidylcholine (HSPC), Hydrogenated Soya lecithin, Dimyristoyl phosphatidyl ethanolamine (DMPE), Dipalmitoyl phosphatidyl ethanolamine (DPPE), Dimyristoyl Phosphatidylcholine (DMPC), Dipalmitoyl Phosphatidylcholine (DPPC), Distearoylphosphatidyl choline (DSPC), Dilauroyl phosphatidylcholine (DLPC), 1-myristoyl-2-palmitoyl phosphatidylcholine, 1-palmitoyl-2-myristoyl phosphatidylcholine, 1-Palmitoyl phosphatidylcholine, 1-stearoyl-2-palmitoyl Phosphatidylcholine, Dipalmitoyl Sphingomyelin, Distearoyl Sphingomyelin, Hydrogenated phosphatidyl inositol (HPI), Dimyristoyl phosphatidyl glycerol (DMPG), Dipalmitoyl phosphatidyl glycerol (DPPG), Distearoyl phosphatidyl glycerol (DSPG), Dimyristoyl phosphatidic acid (DMPA), Dipalmitoyl phosphatidic acid (DPPA), Dimyristoyl phosphatidyl serine (DMPS), Dipalmitoyl phosphatidyl serine (DPPS), Diphosphatidyl glycerol (DPG), Hydrogenated Soya phosphatidyl glycerol (SPG-3), Dioleoyl phosphatidyl glycerol (DOPG), Distearoyl phosphatidic acid (DSPA) and mixtures thereof.

The saturated phospholipids can be employed in the range of between 40 to 50 mole percent of the total concentrates or proliposomal compositions. The saturated phospholipids can be a zwitterionic or anionic in nature. A preferred saturated phospholipid is Hydrogenated Soya Phosphatidyl Choline (HSPC).

The sterol compounds that can be employed as membrane stabilizing agents in the concentrates or proliposomal compositions of the present invention can be selected from the group consisting of Cholesterol, Cholesterol derivatives, Vitamin D, Cholesteryl esters, and mixtures thereof. Cholesterol, in particular, being a major constituent of plasma cell membranes is found to influence the functions of proteins residing in the membrane. Presence of such a sterol in liposomal compositions was found to help in internalisation of the drug. A preferred sterol that can be employed in the composition is Cholesterol.

The sterol compounds can be employed in the range of between 25 to 35 mole percent of the total concentrates or proliposomal compositions. A preferred sterol compound is cholesterol.

In addition, as mentioned hereinbefore, the concentrates or proliposomal compositions of the present invention may optionally contain Polyethylene Glycol (PEG)-coupled lipids. While, not bound by any theory it is probable that the said Polyethylene Glycol (PEG)-coupled lipids either act as membrane stabilizing agents or help in longer circulation of the active principle in the blood stream.

The Polyethylene Glycol (PEG)-coupled lipids that can be employed in the concentrates or proliposomal compositions of the present invention of the present invention are selected from the group consisting of Carbonyl methoxypolyethylene glycol-distearoyl phosphatidyl ethanolamine (MPEG-750-DSPE, -MPEG-2000-DSPE and MPEG-5000-DSPE), Carbonyl methoxypolyethylene glycol-dipalmitoyl phosphatidyl ethanolamine (MPEG-2000-DPPE and MPEG-5000-DPPE), Carbonyl methoxypolyethylene glycol-dimyristoyl phosphatidyl ethanolamine (MPEG-2000-DMPE and MPEG-5000-DMPE) and their derivatives.

The Polyethylene Glycol (PEG)-coupled lipids can be employed in the range of between 2 to 5 mole percent of the total concentrates or proliposomal compositions. A preferred Polyethylene Glycol (PEG)-coupled lipid that can be employed in the composition is -MPEG-2000-DSPE.

Again, as mentioned hereinbefore, the concentrates or proliposomal compositions of the present invention may further optionally contain suitable pharmaceutically acceptable excipients, the role of which can be varied like providing stability to the composition, facilitating optimum drug loading, setting an optimum pH of the composition etc.

Such pharmaceutically acceptable excipients can include antioxidants such as α-Tocopherol or its acetate salt; Vitamin E; β-carotene; Carotenoids, such as α-Carotene, Lycopene (the red colour in tomatoes), Lutein, Zeaxanthine, and the like; buffering agents such as citrate buffer, tris-buffer, phosphate buffer and the like; or acidifying agents, viz. acids, both organic and inorganic, such as citric acid, maleic acid, oxalic acid, succinic acid, tartaric acid, hydrochloric acid, hydrobromic acid, phosphoric acid and the like.

The antioxidants can be employed in the range of between 0.20 to 1.0 mole percent of the total concentrate or proliposomal composition. A preferred antioxidant that can be employed in the composition is α-Tocopherol or its acetate salt.

The vehicles for the concentrates or the proliposomal compositions of the present invention are water-miscible organic solvents. Suitable water-miscible organic solvents that can be employed are selected from aliphatic alcohols, especially ethanol; dialkyl amides, especially dimethylformamide, and dimethylacetamide; dialklyl sulfoxides, especially dimethyl sulfoxide and diethyl sulfoxide; polyethylene glycols of various molecular weights; propylene glycol or mixtures thereof.

The water-miscible organic solvents that can be typically employed as vehicle for concentrates or the proliposomal compositions of the present invention are ethanol, dimethylacetamide, ethanol-polyethylene glycol mixtures, ethanol-propylene glycol mixtures etc. When mixtures of ethanol-polyethylene glycol or ethanol-propylene glycol are employed as vehicles, typically it is preferable to employ them in ratios of 1:1 to 1:0.05 by volume.

Commercially available water-miscible organic solvents can be employed as such for use in the concentrates or proliposomal compositions, or if desired, they can be purified prior to use in the concentrates or proliposomal compositions. The solvents can be purified by methods known in the art. As an example, ethanol and polyols can be purified by pre-treatment with an acid or with an ion exchange resin prior to use.

The concentrates or proliposomal compositions of the poorly water-soluble drugs or compounds as active principles, in turn can be manufactured by a simple and convenient method comprising mixing together the respective proportions of the active principle, the membrane lipids, the membrane stabilizing agent and optionally the Polyethylene Glycol (PEG)-coupled phospholipid and/or the pharmaceutically acceptable excipients in the vehicle, which normally is one or more of a water-miscible organic solvent to obtain a solution, followed by sterile filtration into containers for storage.

In one embodiment, the respective proportions of the membrane forming lipids and the membrane stabilizing compound in an appropriate volume of the vehicle are agitated for a sufficient period of time to obtain a clear solution. The mixing or agitation can be carried out either at room temperature or at elevated temperatures of up to 70° C. After complete dissolution of the membrane forming lipids and the membrane stabilizing agent in the vehicle, the clear solution is cooled to room temperature, to which is added the requisite proportion of the active principle, either in the solid form or as a concentrate in the vehicle used. After thorough mixing, the solution is made up to the desired concentration by dilution with the vehicle and subsequently filtered through micro filters and filled and sealed into appropriate containers or filled into appropriate syringes by methods known in the art, for storage and further use in preparation of liposomal compositions of the poorly water-soluble drugs and compounds.

In an optional embodiment, the respective proportions of the membrane forming lipids, the membrane stabilizing compound, and a Polyethylene Glycol (PEG)-coupled lipid in an appropriate volume of the vehicle are agitated for a sufficient period of time to obtain a clear solution. The mixing or agitation can be carried out either at room temperature or at elevated temperatures of up to 70° C. After complete dissolution of the membrane forming lipids, the membrane stabilizing agent, and the Polyethylene Glycol (PEG)-coupled lipid in the vehicle, the clear solution is cooled to room temperature, to which is added the requisite proportion of the active principle, either in the solid form or as a concentrate in the vehicle used. After thorough mixing, the solution is made up to the desired concentration by dilution with the vehicle and subsequently filtered through micro filters and filled and sealed into appropriate containers or filled into appropriate syringes by methods known in the art, for storage and further use in preparation of liposomal compositions of the poorly water-soluble drugs and compounds.

In another optional embodiment, the respective proportions of the membrane forming lipids and the membrane stabilizing compound in an appropriate volume of the vehicle are agitated for a sufficient period of time to obtain a clear solution. The mixing or agitation can be carried out either at room temperature or at elevated temperatures of up to 70° C. After complete dissolution of the membrane forming lipids and the membrane stabilizing agent in the vehicle, the clear solution is cooled to room temperature, to which is added the requisite proportion of the active principle, either in the solid form or as a concentrate in the vehicle used. After thorough mixing, the pH of the solution, if desired can be adjusted to a suitable range by addition of a buffering agent or an acidifying agent, subsequent to which the solution is made up to the desired concentration by dilution with the vehicle and subsequently filtered through micro filters and filled and sealed into appropriate containers or filled into appropriate syringes by methods known in the art, for storage and further use in preparation of liposomal compositions of the poorly water-soluble drugs and compounds.

In a further optional embodiment, the respective proportions of the membrane forming lipids, the membrane stabilizing compound, and a Polyethylene Glycol (PEG)-coupled lipid in an appropriate volume of the vehicle are agitated for a sufficient period of time to obtain a clear solution. The mixing or agitation can be carried out either at room temperature or at elevated temperatures of up to 70° C. After complete dissolution of the membrane forming lipids, the membrane stabilizing agent, and the Polyethylene Glycol (PEG)-coupled lipid in the vehicle, the clear solution is cooled to room temperature, to which is added the requisite proportion of the active principle, either in the solid form or as a concentrate in the vehicle used. After thorough mixing, the pH of the solution, if desired can be adjusted to a suitable range by addition of a buffering agent or an acidifying agent, subsequent to which the solution is made up to the desired concentration by dilution with the vehicle and subsequently filtered through micro filters and filled and sealed into appropriate containers or filled into appropriate syringes by methods known in the art, for storage and further use in preparation of liposomal compositions of the poorly water-soluble drugs and compounds.

As would be evident, the method(s) do(es) not call for adherence to any critical parameter or operation and thereby does away with any critical supervision and moreover, does not require any skill or dexterity on the part of the operator for manufacture of the object concentrates or proliposomal compositions.

Further, as mentioned hereinbefore, the concentrates or proliposomal compositions of the poorly water-soluble drugs and compounds, thus prepared were found to be stable for at least 3 to 6 months at 25±2° C. and at 60±5% RH and at 2-8° C., with reasonable to no drop in assay of the active principle from the initial value. The compositions remained clear, without any observable sedimentation for the three to six month period they were observed.

Specifically, a 3 to 6 month stability profile of the concentrate or proliposomal composition of the anticancer drug, Docetaxel is summarized in Table-I, which should be considered as only as an exemplifying embodiment and in no way should be construed as limiting the scope of the invention.

Furthermore, as mentioned hereinbefore, the other advantage the concentrates or proliposomal compositions of the present invention offer is that by virtue of their enhanced stability, even at ambient or refrigeration temperatures, the said concentrates or compositions could be stored for prolonged period of time, without significant loss in potency of the active principle and also could be transported under such storage conditions in a more convenient manner, which moreover, significantly brings down the cost of transportation as well storage in warehouses.

### The Liposomal Compositions of Poorly Water-Soluble Drugs and Compounds of the Present Invention

The concentrates or proliposomal compositions of poorly water-soluble drugs or compounds, as discussed and obtained hereinbefore, could be conveniently utilized for formation, preparation, or manufacture of liposomal compositions of poorly water-soluble drugs or compounds instantly at the bedside of patients in need of treatment or administration of the said poorly water-soluble drugs or compounds, through a simple operation of injection of the said concentrates or proliposomal compositions into a suitable diluting fluid for administration, which can be carried out safely by a practicing doctor or other qualified medical or paramedical supervisors or staff.

TABLE-I
Stability Of The Concentrate Or Proliposomal Composition Of
Docetaxel As Per The Present Invention

| | | | | Assay of Docetaxel | | | | |
|---|---|---|---|---|---|---|---|---|
| Sr. | | Qty | | (mg/ml) | | | | |
| No. | Unit Composition | (mg) | Condition | Initial | 1M | 2M | 3M | 6M |
| 1 | Docetaxel | 9 | 25 ± 2° C./ | 9.5 | 9.4 | 9.3 | 9.3 | 9.1 |
| | HSPC | 37.5 | 60 ± 5% RH | | | | | |
| | Cholesterol | 11.25 | | | | | | |
| | EPG | 15 | 2-8° C. | 9.5 | 9.4 | 9.4 | 9.4 | 9.1 |
| | Ethanol (q.s.) | 1 ml | | | | | | |
| 2 | Docetaxel | 9 | 25 ± 2° C./ | 9.2 | 8.9 | 8.9 | 8.6 | 8.7 |
| | HSPC | 37.5 | 60 ± 5% RH | | | | | |
| | Cholesterol | 11.25 | | | | | | |
| | EPG | 15 | 2-8° C. | 9.2 | 9 | 9 | 8.9 | 8.8 |
| | α Tocopherol | 0.5 | | | | | | |
| | Ethanol (q.s.) | 1 ml | | | | | | |
| 3 | Docetaxel | 9 | 25 ± 2° C./ | 8.8 | 8.9 | 8.8 | 8.9 | — |
| | HSPC | 37.5 | 60 ± 5% RH | | | | | |
| | Cholesterol | 11.25 | | | | | | |
| | EPG | 15 | 2-8° C. | 8.8 | 8.9 | 8.9 | 8.8 | — |
| | α Tocopherol | 0.5 | | | | | | |
| | Ethanol + PG* | 1 ml | | | | | | |
| | (9:1, q.s.) | | | | | | | |
| 4 | Docetaxel | 9 | 25 ± 2° C./ | 9.1 | 8.9 | 9 | 8.8 | 8.7 |
| | HSPC | 37.5 | 60 ± 5% RH | | | | | |
| | Cholesterol | 11.25 | | | | | | |
| | EPG | 15 | | | | | | |
| | α Tocopherol | 0.5 | 2-8° C. | 9.1 | 8.9 | 9 | 8.6 | 8.7 |
| | MPEG2000- | 7.5 | | | | | | |
| | DSPE | | | | | | | |
| | Ethanol (q.s.)_{—} | 1 ml | | | | | | |

*PG = Propylene Glycol

The liposomes can be formed instantly on injection of the concentrates or proliposomal compositions into the diluting fluid. While, there could be some variation in the mean particle size diameter of the liposomes so formed, however, it is an aspect of the present invention that liposomes of consistent particle size diameter of less than 100 nm can be obtained, produced, or manufactured in the diluting fluid for reconstitution by injection of the concentrates or proliposomal compositions, through syringes with hypodermic needles having a gauge of between 18 G to 30 G, at a rate of about 0.10 ml/second to about 1.5 ml/second. Further the degree of entrapment or encapsulation of the poorly water-soluble drugs or compounds in the liposomes was found to be very high and in most instances it was found to be ≧95%.

The liposomes thus obtained, produced, or manufactured in the diluting fluid for reconstitution, apart from having the advantage of being obtained, produced, or manufactured in consistent particle size diameter of less than 100 nm in most instances, are found to possess significantly higher physical stability in the reconstitution medium, for instance a physical stability of not less than 4 hours and in many instances ≧24 hours, depending of the nature of the poorly water-soluble drug or compound entrapped or encapsulated in the liposomes.

A liposomal composition of the anticancer drug, Docetaxel, prepared by injection, of a concentrate or proliposomal composition of the same in a mole percent of between 9 to 11, comprising of Hydrogenated soy phosphatidyl choline (HSPC) as the saturated membrane forming lipid in a mole percent of between 44 to 46, Egg Phosphatidyl Glycerol (EPG) as the unsaturated membrane forming lipid in a mole percent of between 16-18, and cholesterol as the membrane stabilizing agent in a mole percent of between 26 to 27, into a 5% Dextrose solution as the diluting fluid, through syringes with hypodermic needles having a gauge of between 18 G to 30 G, at a rate of about 0.10 ml/second to about 1.5 ml/second, was found to have a particle size diameter of about 95 nm and having a physical stability of more than 12 hours, with no crystallization or precipitation of the drug from the reconstituted media. Further, the entrapment or encapsulation of the drug in the liposomes was found to be greater than 95%. This specific embodiment should be considered as only as an exemplifying embodiment and in no way should be construed as limiting the scope of the invention.

It might be mentioned herein that Docetaxel, is an anticancer drug, first disclosed in U.S. Pat. No. 4,814,470. While many forms of Docetaxel are known, like the crystalline anhydrous, crystalline hemihydrate, and crystalline trihydrate and all these “Crystalline Forms” can be utilized as the poorly water-soluble drug or compound for preparation of the concentrate or proliposomal composition of the present invention, however, it is found advantageous to use an “Amorphous Form” of Docetaxel in the present invention. Such an “Amorphous Form” of Docetaxel and its preparation are disclosed in our Pending Indian Application No. 253/Kol/2007.

Similarly, liposomal compositions of other poorly water-soluble drugs and compounds could be prepared from the corresponding concentrates or proliposomal compositions and can be obtained in particle size diameter of less than 100 nm, employing the same technique. For example, a liposomal composition of the anticancer drug, Paclitaxel can be prepared with about 95% entrapment or encapsulation of the drug within the liposome in particle size diameter in the range of 90 nm and further having a physical stability of >5 hours; a liposomal composition of the Betulinic acid derivative, MJ-1098 (I) can be prepared with about 95% entrapment or encapsulation of the drug within the liposome in particle size diameter in the range of about 90 nm and further having a physical stability of >6 hours; a liposomal composition of the Betulinic acid derivative, DRF-4012 (II) can be prepared with about 95% entrapment or encapsulation of the drug within the liposome in particle size diameter in the range of about 90 nm and further having a physical stability of >6 hours; a liposomal composition of the Betulinic acid derivative, DRF-4015 (III) can be prepared with about 95% entrapment or encapsulation of the drug within the liposome in particle size diameter in the range of 95 nm and further having a physical stability of >6 hours; and a liposomal composition of the immunomodulator, Cyclosporine can be prepared with about 95% entrapment or encapsulation of the drug within the liposome in particle size diameter in the range of about 95 nm and further having a physical stability of >24 hours. Here again, embodiments should be considered as only as an exemplifying embodiment and in no way should be construed as limiting the scope of the invention.

In one embodiment, the concentrates or proliposomal compositions of poorly water-soluble drugs and compounds, contained in sealed glass vials or vials made up of other non-toxic materials, is withdrawn into a syringe, with a hypodermic needle of gauge 18 G to 30 G. The withdrawn concentrates or proliposomal compositions is then injected rapidly, at a rate of about 0.10 ml/second to about 1.5 ml/second into the container containing the diluting fluid, with the tip of the needle extended below the surface of the diluting fluid. After complete injection of the concentrates or proliposomal compositions, the mixture is shaken gently for a few minutes to obtain a uniform dispersion of the liposomes of the poorly water-soluble drugs or compounds, which is then ready for administration to patients in need thereof.

Suitable vials made of non-toxic materials other than glass include vials constructed of materials like plastic, polypropylene, polyethylene, polyesters, polyamides, polycarbonates, hydrocarbon polymers etc.

In another embodiment, the concentrates or proliposomal compositions of poorly water-soluble drugs and compounds, contained in a pre-filled syringe, fitted with a hypodermic needle having a gauge of 18 G to 30 G is then injected rapidly, at a rate of about 0.10 ml/second to about 1.5 ml/second into the container containing the diluting fluid, with the tip of the needle extended below the surface of the diluting fluid. After complete injection of the concentrates or proliposomal compositions, the mixture is shaken gently for a few minutes to obtain a uniform dispersion of the liposomes of the poorly water-soluble drugs or compounds, which is then ready for administration to patients in need thereof.

While, utilization of rate of injection of the concentrates or proliposomal compositions into the diluting fluid other than the specified rate of about 0.10 ml/second to about 1.5 ml/second or utilization of hypodermic needles of gauges, different from that of 18 G to 30 G for injection of the concentrates or proliposomal compositions into the diluting fluid, are not highly preferred in terms of obtaining the liposomes having particle size diameters of less than 100 nm as well as having optimum physical stability, nevertheless, utilization of the same also leads to formation of the liposomes, albeit in particle size diameters higher than 100 nm as well as having physical stability less than 4 hours, the reason why utilization of a rate of injection of about 0.10 ml/second to about 1.5 ml/second and hypodermic needles of gauges 18 G to 30 G are preferred.

Suitable diluting fluids that can be employed for reconstitution of the concentrates or proliposomal compositions and preparation of the liposomal compositions can be selected from, but not limited to water, saline, 5% and 10% dextrose solutions, dextrose and sodium chloride solution, sodium lactate solution, lactated Ringer solution, mannitol solution, mannitol with dextrose or sodium chloride solution, Ringer's solution, sterile water for injection and multiple electrolyte solutions comprising varying combinations of electrolytes, dextrose, fructose and invert sugar. However, a preferred diluting fluid is a fluid comprising dextrose and water and more preferably 5% and 10% dextrose solutions.

### Non-Clinical Studies on a Liposomal Composition of the Anticancer Drug, Docetaxel, Prepared as Per the Method of the Present Invention

Discussed hereinbelow are some of the non-clinical studies carried out by the present inventors on a liposomal composition of the anticancer drug, Docetaxel, prepared as per the method of the present invention, the details of which have been discussed in detail hereinbefore.

As mentioned hereinbefore, in all the studies an “Amorphous Form” of Docetaxel is used, ad disclosed in our Pending Indian Application No. 253/Kol/2007.

The non-clinical studies carried out include determination of the pharmacodynamics including cytotoxicity and tubulin polymerization activity, efficacy, pharmacokinetics, and safety.

In all the studies, wherever a comparison of the abovementioned studies was required with a conventional, approved and marketed composition of Docetaxel, the one marketed by M/S Sanofi-Aventis under the brand name, Taxotere® was used.

### 1.0 Pharmacology

### 1.1 Primary Pharmacodynamics

### 1.1.1 In Vitro Cytotoxicity

The cytotoxicity of the Liposomal composition of Docetaxel (hereinafter referred to as “LD”) in vitro in a panel of human cancer cell lines, expected to be sensitive to Docetaxel and the effects were compared with the conventional, approved, marketed composition of Docetaxel, viz. Taxotere® (hereinafter referred to as “CD”). A solution of bulk Docetaxel in DMSO was taken as a positive control for the studies.

The growth inhibition (IC₅₀) of both the formulations were in the low nanomolar range in human ovary, prostate, and breast cancer cell lines in a 72 hour MIT assay. Data, summarized in Table-II suggests that the spectrum of activity of LD was comparable to that of the CD.

### 1.1.2 In Vivo Anti-Tumour Effects

An efficacy study was conducted to compare the anti-tumour activity of LD with CD, when administered to C57Bl/6 mice, bearing Murine Melanoma (B16F10) tumour xenograft, by intravenous route.

TABLE II
in vitro IC₅₀ of LD and CD

| | | IC₅₀ Values (nM) | | |
|---|---|---|---|---|
| | | | | Docetaxel |
| Tumour | | | | Solution in |
| Type | Cell Line | CD | LD | DMSO |
| Breast | MDA MB 453 | 18.30 ± 2.30 | 14.05 ± 3.20 | 15.07 ± 2.30 |
| Ovary | PAI | <0.01 | <0.01 | <0.01 |
| | SKOV3 | 10.56 ± 1.90 | 12.70 ± 2.34 | 9.87 ± 2.74 |
| Prostrate | DU145 | 2.93 ± 1.39 | 5.28 ± 2.69 | 5.13 ± 1.28 |

Female C57BL/6 mice 6-8 weeks of age and weighing 20-25 g were used for the study. There were 7 animals in each of the treated group and 6 animals in the control group. The animals were acclimatized for a period of one week prior to the start of treatment. LD and CD were administered at a dose of 24 mg/kg. Control group received equivalent volume of 5% dextrose corresponding to the highest dose. The test substances were administered on 3^{rd}, 5^{th}, 7^{th} and 9^{th} day post inoculation of the tumour cells using sterile 1 ml disposable syringe and 30 G needle. The animals were observed for signs of toxicity, tumour reduction, body weight and mortality. At the conclusion of study, all the surviving animals were sacrificed, tumours were excised and their weights measured.

The regression of tumour due to treatment is described in terms of Treated/Control (T/C) %, which is defined as follows:

$$T / C \% = \frac{Change in tumour {volume}_{treated}}{Change in tumour {volume}_{control}} \times 100$$

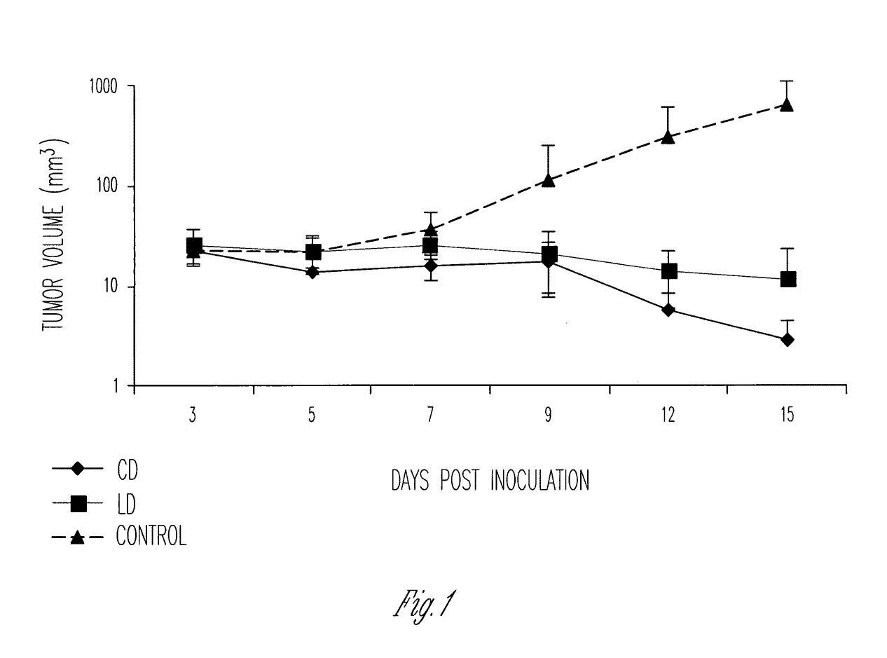

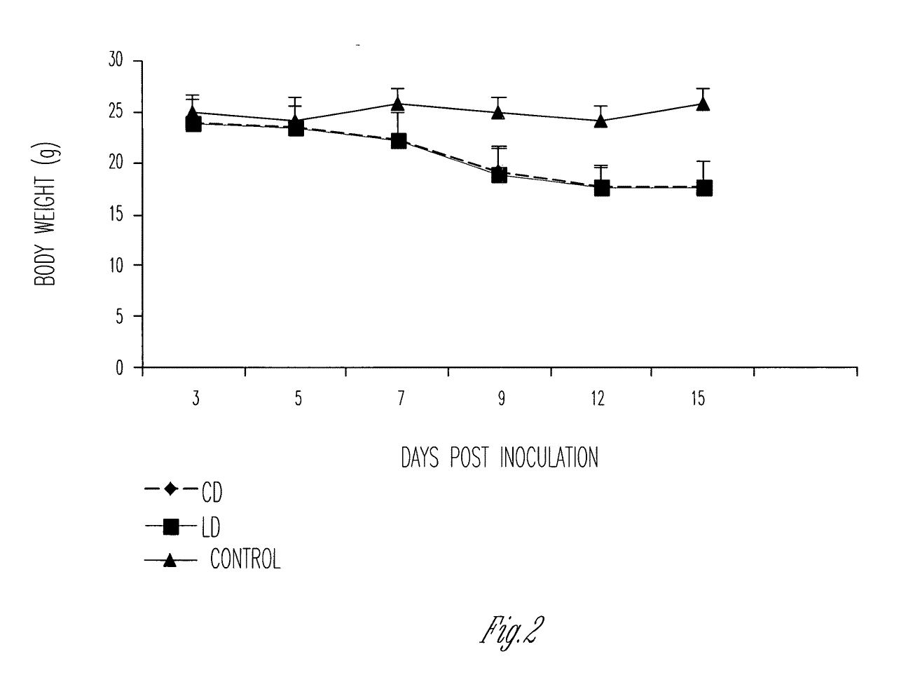

The tumour volumes of LD vs. CD treated groups are given in Table-III. FIG. 1 shows the kinetics of tumour regression while FIG. 2 shows the body weight of animals over the treatment period.

Mice treated LD exhibited T/C of 2.3% as compared those treated with CD, which showed a T/C value of 3.1%. A T/C of less than 42% is considered significant. There were no abnormal clinical signs in any animal in all the groups. After the excision of tumours on 15^{th} days based on tumour weights, the median T/C value was observed to be 0.6% in LD treated mice and 0.5% in CD treated mice.

Hence, the two formulations were found to cause a comparable tumour regression activity.

TABLE III
Comparison Of Tumour Volumes Of LD and CD
When Administered to C57bl/6 Mice Bearing Murine
Melanoma By Intravenous Route*
Tumour Volume

| Test | | | | | | |
|---|---|---|---|---|---|---|
| Substance | LD | | CD | | Control | |
| Days | Mean | SD | Mean | SD | Mean | SD |
| 3 | 26.6 | 9.5 | 22.3 | 6.7 | 21.1 | 6.4 |
| 5 | 22.2 | 8.4 | 14.1 | 1.1 | 21.1 | 10.1 |
| 7 | 26.5 | 9.4 | 16.1 | 4.5 | 36.3 | 18.4 |
| 9 | 21.4 | 13 | 17.4 | 9.8 | 115 | 142.1 |
| 12 | 14.2 | 8.1 | 5.8 | 2.7 | 307.2 | 294.7 |
| 15 | 11.7 | 12.0 | 2.8 | 1.6 | 649.9 | 476.3 |

*Measurement day Post Inoculation

Mice treated LD exhibited T/C of 2.3% as compared those treated with CD, which showed a T/C value of 3.1%. A T/C of less than 42% is considered significant. There were no abnormal clinical signs in any animal in all the groups. After the excision of tumours on 15^{th} day, based on tumour weights, the median T/C value was observed to be 0.6% in LD treated mice and 0.5% in CD treated mice.

Hence, the two formulations were found to cause a comparable tumour regression activity.

### 2.0 Secondary Pharmacodynamics

### 2.1 Tubulin Polymerization

The Pharmacodynamics of LD was evaluated by quantitation of tubulin polymerization potential in ovarian carcinoma cells (PA1 cells) and the effects were compared with that of CD. The cells were treated with 0.01-100 nM of LD or CD and harvested after 17 hours of incubation. To assess the time-kinetics, the cells were treated with 1 uM of either LD or CD and harvested at specific time intervals varying from 15-120 minutes. The cells were lysed in hypotonic buffer conditions. The soluble and polymerized tubulin was separated by centrifugation. Pellets and supernatants were processed separately and analyzed by polyacrylamide gel electrophoresis, followed by transfer onto a PVDF membrane and finally immunoblotting using primary anti-alpha-tubulin antibody. Expression of soluble and polymerized tubulin were quantified by densitometry using the public domain NIH image program and percentage of polymerized tubulin was measured and dose and time response curves were plotted.

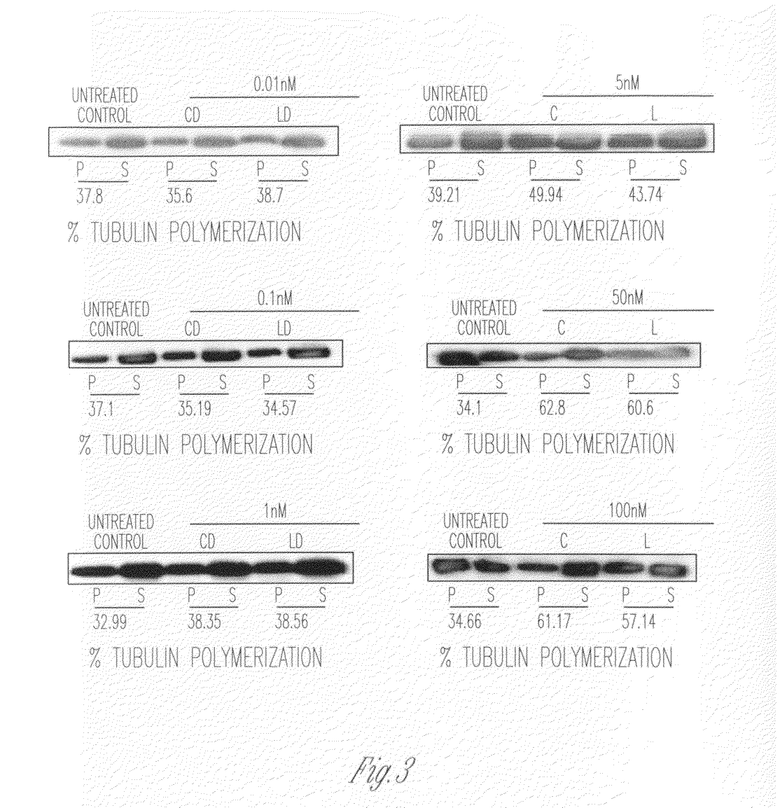

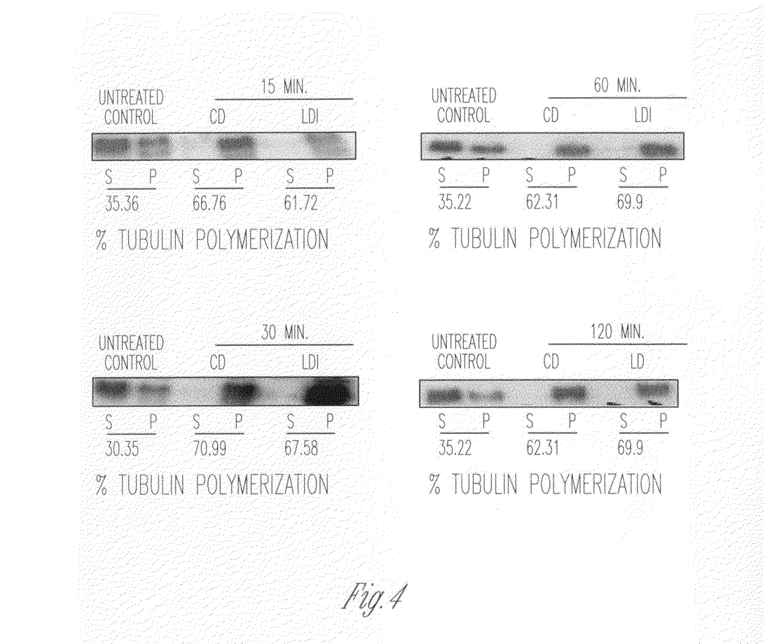

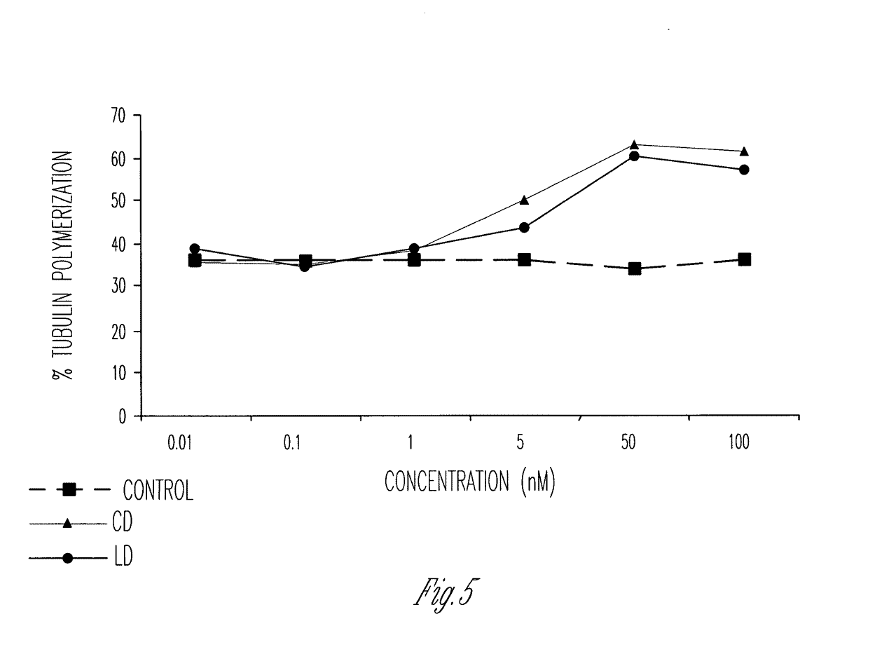

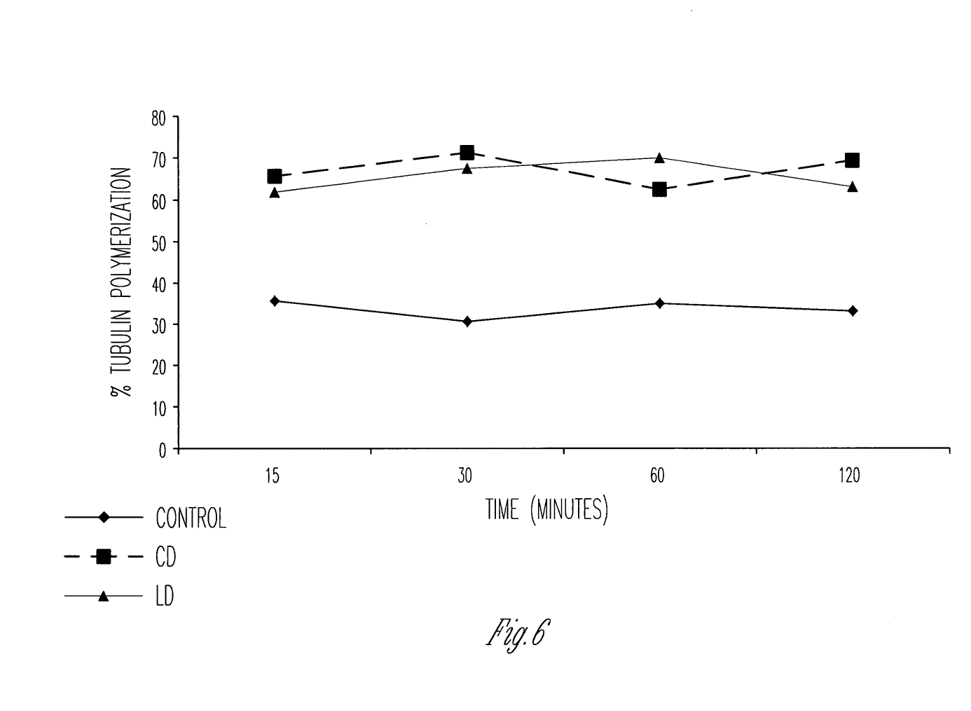

The study suggested that Docetaxel retains the tubulin binding property after liposome encapsulation and the extent of tubulin polymerization in ovarian cancer cells was comparable to that observed in the conventional composition (CD). FIG. 3 and FIG. 4 depict the dose and time kinetics data for tubulin polymerization in PA1 cell line respectively. The dose and time dependent effects on tubulin polymerization are shown graphically in FIGS. 5 and 6, respectively

### 3.0 Pharmacokinetics

Pharmacokinetics of LD and CD were compared in this study. The Pharmacokinetic study was conducted in Female wistar rats, 6-8 weeks of age and weighing approximately 150 gm. Care and handling of animals were in accordance with Institutional Animal Ethics Committee (IAEC). Each one of the composition i.e. LD and CD before administration were suitably diluted with physiological buffer to the desired concentration. Each composition was injected into the group of six animals separately as bolus injection vial the tail vein at doses of 2.5, 5.0 and 10.0 mg/kg.

Blood samples were taken from the retro-orbital plexus at various time points Plasma was separated immediately by centrifugation and stored at −20° C. prior to analysis. Docetaxel from the plasma was extracted via Liquid-Liquid Extraction and analyzed using Liquid Chromatography Mass Spectrometry (LC-MS/MS) technique. Pharmacokinetic parameters were determined using the WinNonlin software 5.2 (Pharsight Corporation). Non-compartment model was used to fit the data. Distribution and elimination were represented by the following parameters area under the curve (AUCₐₗₗ), total body clearance (Cl_{obs}), apparent volume of distribution (V_{d}) and plasma half life (T_{1/2}).

As the C₀, AUCₐₗₗ, T_{1/2} values at each dose of the two compositions are comparable, Pharmacokinetics of the said two compositions can be concluded to be comparable across the doses, the details of which are summarized in Table-IV. Both LD and CD show good linearity with respect to AUC at three doses, with r² value of >0.95 and 0.99 respectively. Other Pharmacokinetic parameters like V_{d}, Cl_{obs} and MRTₗₐₛₜ are also found comparable, as would be evident from Table-IV.

TABLE-IV
Dose Dependent Pharmacokinetic Studies on LD And CD

| | | 2.5 mg/kg | | 5.0 mg/kg | | 10 mg/kg | |
|---|---|---|---|---|---|---|---|
| Parameter | Units | LD | CD | LD | CD | LD | CD |
| C₀ | μg/ml | 0.904 ± 0.14 | 0.917 ± 0.12 | 1.939 ± 0.41 | 3.6745 ± 1.60 | 5.931 ± 2.83 | 5.508 ± 1.55 |
| AUCₐₗₗ | hr * μg/ml | 0.294 ± 0.05 | 0.248 ± 0.04 | 0.6207 ± 0.11 | 0.8646 ± 0.13 | 2.84 ± 1.02 | 2.45 ± 0.39 |
| T_{1/2} | Hr | 3.337 ± 0.43 | 3.952 ± 1.07 | 2.357 ± 0.34 | 4.228 ± 2.57 | 6.671 ± 2.59 | 4.710 ± 2.11 |
| V_{d} | ml/kg | 45.38 ± 9.43 | 56.69 ± 11.44 | 28.83 ± 5.92 | 35.91 ± 20.52 | 38.68 ± 26.75 | 27.91 ± 12.40 |
| Cl_{obs} | ml/hr/kg | 9.421 ± 1.45 | 10.115 ± 0.99 | 8.446 ± 0.85 | 6.016 ± 0.91 | 3.704 ± 1.17 | 4.214 ± 0.72 |

### 4.0 Toxicology

Preclinical toxicity studies are an integral part of safety assessment of a drug and provide a preliminary picture of the toxicity profile of a drug. Sub-acute toxicity studies were carried out to determine the potential toxic effects of LD.

### 4.1 Sub Acute Toxicity

A sub-acute study was conducted to compare the toxicity profile of LD with CD in rodents.

Male/Female Wistar rats, 7-10 weeks of age and weighing 130-275 g (males), 140-180 g (females) and Male/Female Swiss Albino mice, 8-10 weeks of age and weighing 23-35 g were used for the study. There were 5 animals per sex per group. The animals were acclimatized for a period of one week prior to the start of treatment. LD and CD were administered at dose levels of 1.0, 2.5, and 5.0 mg/kg in Wistar rats and a dose of 6.25, 12.5 and 25 mg/kg were administered to Swiss Albino Mice. Controls consisted of a Vehicle group, which comprised the excipients used in compositions (composition minus drug) corresponding to the highest dose. Control group received equivalent volume of 5% dextrose (corresponding to the highest dose). The test substances were administered once every day for 5 continuous days using sterile 1 ml disposable syringe and 30 G needles. Observations comprised of mortality, clinical signs, body weight, food and water consumption, clinical laboratory investigations, organ weights and macroscopic histopathology.

100% mortality was observed in both male and female wistar rats treated with 5.0 mg/kg of both the compositions during the course of the study. All the wistar rats that died exhibited severe watery diarrhoea and a body weight loss terminating in death, 5-7 days post drug administration. Based on the clinical signs observed in these animals the deaths are attributed to the treatment. 40% mortality was observed in animals treated with 2.5 mg/kg. There were no observable clinical signs and treatment mortalities in animals treated with 1 mg/kg, vehicle and dextrose. Dose dependent increase in stomatitis, alopecia, hand and foot syndrome and facial edema was present in both males and females treated with 2.5 mg/kg and 5.0 mg/kg doses, which is a usual finding during treatment with anticancer drugs. There were no other abnormal clinical signs in any animal of the other group.

100% mortality was observed in both male and female swiss albino mice treated with 25.0 mg/kg of both the compositions during the course of the study. Based on the clinical signs observed in these animals the deaths are attributed to the treatment. 40% mortality was observed in animals treated with 12.5 mg/kg. Alopecia, facial edema and paresis/loss of hindlimb extension were observed in groups treated with 25 mg/kg of both the compositions. There were no other abnormal clinical signs in any animal of the other group.

Except for the animals (Wistar rats and swiss albino mice) in the highest and middle dose group, where mortality was observed, animals from all groups of both sex showed a progressive increase in body weight during the course of the study.

Dose dependent decrease in food and water consumption was noticed in both the species during the study. Dose dependent decrease in neutrophil count and total leucocyte count was observed in both the species, either sex for both the compositions. The hematological parameters in the animal groups treated with dextrose and vehicle were within the normal. The Highest Non Toxic Dose (HNTD) was found to be 5 mg/Kg (1 mg/Kg×5 days) in Wistar Rats for both the compositions. In swiss albino mice the Highest Non Toxic Dose (HNTD) was found to be 31.25 mg/Kg (6.25 mg/Kg×5 days) in both the compositions.

Hence, both compositions i.e. LD and CD can be concluded to demonstrate similar toxicity profiles.

The invention is further illustrated by way of the following examples, which in no way should be construed as limiting to the scope of the invention.

### EXAMPLE 1

### Liposomal Composition of Docetaxel

### Step-1: Preparation of Concentrate or Proliposomal Composition

50 mg of Hydrogenated Soya phosphatidyl choline (HSPC, 45.01 mole %), 15 mg Cholesterol (26.61 mole %), 20 mg Egg phosphatidyl glycerol (EPG, 17.79 mole %), and 0.15 mg of α-Tocopheryl acetate (0.22 mole %) were dissolved in 1 ml of absolute ethanol which was then heated at 70° C. for 2 minutes using water bath to obtain a clear solution of lipids. The solution was brought down to room temperature, to which was added 12 mg of amorphous Docetaxel (10.37 mole %). The Concentrate or Proliposomal Composition of Docetaxel so obtained was mixed using magnetic stirrer/vortex shaker until clear. The solution thus obtained was filtered through 0.22 μm filters.

### Step-2: Preparation of Liposomal Composition

0.5 ml of the Concentrate or Proliposomal Composition of Docetaxel, as obtained in Step-1 was rapidly injected at a rate of 0.16 ml/second using a 1 ml syringe with a hypodermic needle of gauge 30 G into 7.5 ml of 5% Dextrose solution to obtain a dispersion containing Docetaxel loaded liposomes, providing the object Liposomal Composition of Docetaxel at a drug concentration of 0.75 mg/ml.

The liposomal composition thus prepared had a particle size of approximately 90 nm and a stability of more than 10 hours

### EXAMPLE 2

### Liposomal Composition of Docetaxel

### Step-1: Preparation of Concentrate or Proliposomal Composition

50 mg of Hydrogenated Soya phosphatidyl choline (HSPC, 45.01 mole %), 15 mg Cholesterol (26.61 mole %), 20 mg Egg phosphatidyl glycerol (EPG, 17.79 mole %), and 0.15 mg of α-Tocopheryl acetate (0.22 mole %) were dissolved in 1 ml of a mixture of absolute ethanol and propylene glycol (9:1 ratio), which was then heated at 70° C. for 2 minutes using water bath to obtain a clear solution of lipids. The solution was brought down to room temperature, to which was added 12 mg of amorphous Docetaxel (10.37 mole %). The Concentrate or Proliposomal Composition of Docetaxel so obtained was mixed using magnetic stirrer/vortex shaker until clear. The solution thus obtained was filtered through 0.22 μm filters.

### Step-2: Preparation of Liposomal Composition

0.5 ml of the Concentrate or Proliposomal Composition of Docetaxel, as obtained in Step-1 was rapidly injected at a rate of 0.12 ml/second using a 1 ml syringe with a hypodermic needle of gauge 29 G into 7.5 ml of 5% Dextrose solution to obtain a dispersion containing Docetaxel loaded liposomes, providing the object Liposomal Composition of Docetaxel at a drug concentration of 0.75 mg/ml.

The liposomal composition thus prepared had a particle size of approximately 95 nm and a stability of more than 10 hours.

### EXAMPLE 3

### Liposomal Composition of Docetaxel

### Step-1: Preparation of Concentrate or Proliposomal Composition

50 mg of Hydrogenated Soya phosphatidyl choline (HSPC, 45.19 mole %), 15 mg Cholesterol (26.73 mole %) and 20 mg Egg phosphatidyl glycerol (EPG, 17.84 mole %) were dissolved in 1 ml of absolute ethanol which was then heated at 70° C. for 2 minutes using water bath to obtain a clear solution of lipids. The solution was brought down to room temperature. 12 mg of amorphous Docetaxel (10.23 mole %) was then added to this solution. The Concentrate or Proliposomal Composition of Docetaxel so obtained was mixed using magnetic stirrer/vortex shaker until clear. The solution thus obtained was filtered through 0.22 μm filters.

### Step-2: Preparation of Liposomal Composition

0.5 ml of the Concentrate or Proliposomal Composition of Docetaxel, as obtained in Step-1 was rapidly injected at a rate of 0.10 ml/second using a 1 ml syringe with a hypodermic needle of gauge 30 G into 7.5 ml of 5% Dextrose solution to obtain a dispersion containing Docetaxel loaded liposomes, providing the object Liposomal Composition of Docetaxel at a drug concentration of 0.75 mg/ml.

The liposomal composition thus prepared had a particle size of approximately 95 nm and a stability of more than 12 hours.

### EXAMPLE 4

### Liposomal Composition of Docetaxel

### Step-1: Preparation of Concentrate or Proliposomal Composition

50 mg of Hydrogenated Soya phosphatidyl choline (HSPC, 45.19 mole %), 15 mg Cholesterol (26.73 mole %) and 20 mg Egg phosphatidyl glycerol (EPG, 17.84 mole %) were dissolved in 1 ml of a mixture of absolute ethanol and Propylene glycol (9:1) which was then heated at 70° C. for 2 minutes using water bath to obtain a clear solution of lipids. The solution was brought down to room temperature. 12 mg of amorphous Docetaxel (10.23 mole %) was then added to this solution. The Concentrate or Proliposomal Composition of Docetaxel so obtained was mixed using magnetic stirrer/vortex shaker until clear. The solution thus obtained was filtered through 0.22 μm filters.

### Step-2: Preparation of Liposomal Composition

0.5 ml of the Concentrate or Proliposomal Composition of Docetaxel, as obtained in Step-1 was rapidly injected at a rate of 0.16 ml/second using a 1 ml syringe with a hypodermic needle of gauge 28 G into 7.5 ml of 5% Dextrose solution to obtain a dispersion containing Docetaxel loaded liposomes, providing the object Liposomal Composition of Docetaxel at a drug concentration of 0.75 mg/ml.

The liposomal composition thus prepared had a particle size of approximately 98 nm and a stability of more than 12 hours.

### EXAMPLE 5

### Liposomal Composition of Docetaxel

### Step-1: Preparation of Concentrate or Proliposomal Composition

37.5 mg of Hydrogenated Soya phosphatidyl choline (HSPC, 45.16 mole %), 11.25 mg Cholesterol (26.71 mole %) and 15 mg Egg phosphatidyl glycerol (EPG, 17.89 mole 0%) were dissolved in 1 ml of a mixture of absolute ethanol and Propylene glycol (9:1) which was then heated at 70° C. for 2 minutes using water bath to obtain a clear solution of lipids. The solution was brought down to room temperature. 9 mg of amorphous Docetaxel (10.22 mole %) was then added to this solution. The Concentrate or Proliposomal Composition of Docetaxel so obtained was mixed using magnetic stirrer/vortex shaker until clear. The solution thus obtained was filtered through 0.22 μm filters.

### Step-2: Preparation of Liposomal Composition

1.0 ml of the Concentrate or Proliposomal Composition of Docetaxel, as obtained in Step-1 was rapidly injected at a rate of 0.25 ml/second—using a 1 ml syringe with a hypodermic needle of gauge 30 G into 111 ml of 5% Dextrose solution to obtain a dispersion containing Docetaxel loaded liposomes, providing the object Liposomal Composition of Docetaxel at a drug concentration of 0.75 mg/ml.

The liposomal composition thus prepared had a particle size of approximately 85 nm and a stability of more than 12 hours.

### EXAMPLE 6

### Liposomal Composition of Docetaxel

### Step-1: Preparation of Concentrate or Proliposomal Composition

37.5 mg of Hydrogenated Soya phosphatidyl choline (HSPC, 44.71 mole %), 11.25 mg Cholesterol (26.44 mole %), 15 mg Egg phosphatidyl glycerol (EPG, 17.72 mole %) and 0.5 mg of α-Tocopherol (1.0 mole %) were dissolved in 1 ml of a mixture of absolute ethanol and Propylene glycol (9:1) which was then heated at 70° C. for 2 minutes using water bath to obtain a clear solution of lipids. The solution was brought down to room temperature. 9 mg of amorphous Docetaxel (10.12 mole %) was then added to this solution. The Concentrate or Proliposomal Composition of Docetaxel so obtained was mixed using magnetic stirrer/vortex shaker until clear. The solution thus obtained was filtered through 0.22 μm filters.

### Step-2: Preparation of Liposomal Composition

1.0 ml of the Concentrate or Proliposomal Composition of Docetaxel, as obtained in Step-1 was rapidly injected at a rate of 0.20 ml/second using a 1 ml syringe with a hypodermic needle of gauge 26 G into 11 ml of 5% Dextrose solution to obtain a dispersion containing Docetaxel loaded liposomes, providing the object Liposomal Composition of Docetaxel at a drug concentration of 0.75 mg/ml.

The liposomal composition thus prepared had a particle size of approximately 100 nm and a stability of more than 10 hours.

### EXAMPLE 7

### Liposomal Composition of Docetaxel

### Step-1: Preparation of Concentrate or Proliposomal Composition

37.5 mg of Hydrogenated Soya phosphatidyl choline (HSPC, 45.16 mole %), 11.25 mg Cholesterol (26.71 mole %) and 15 mg Egg phosphatidyl glycerol (EPG, 17.89 mole %) were dissolved in 1 ml of absolute ethanol which was then heated at 70° C. for 2 minutes using water bath to obtain a clear solution of lipids. The solution was brought down to room temperature. 9 mg of amorphous Docetaxel (10.22 mole %) was then added to this solution. The Concentrate or Proliposomal Composition of Docetaxel solution so obtained was mixed using magnetic stirrer/vortex shaker until clear. The solution thus obtained was filtered through 0.22 μm filters.

### Step-2: Preparation of Liposomal Composition

1.0 ml of the Concentrate or Proliposomal Composition of Docetaxel, as obtained in Step-1 was rapidly injected at a rate of 0.5 ml/second using a 1 ml syringe with a hypodermic needle of gauge 20 G into 11 ml of 5% Dextrose solution to obtain a dispersion containing Docetaxel loaded liposomes, providing the object Liposomal Composition of Docetaxel at a drug concentration of 0.75 mg/ml.

The liposomal composition thus prepared had a particle size of approximately 95 nm and a stability of more than 8 hours.

### EXAMPLE-8

### Liposomal Composition of Docetaxel

### Step-1: Preparation of Concentrate or Proliposomal Composition

50 mg of Hydrogenated Soya phosphatidyl choline (HSPC, 43.89 mole %), 15 mg Cholesterol (25.95 mole %), 20 mg Egg phosphatidyl glycerol (EPG, 17.35 mole %), 10 mg of Carbonyl methoxy polyethylene glycol 2000-distearoyl phosphatidyl ethanolamine (2.48 mole %) and 0.15 mg of α-Tocopheryl acetate (0.21 mole %) were dissolved in 1 ml of absolute ethanol which was then heated at 70° C. for 2 minutes using water bath to obtain a clear solution of lipids. The solution was brought down to room temperature. 12 mg of amorphous Docetaxel (10.11 mole %) was then added to this solution. The Concentrate or Proliposomal Composition of Docetaxel so obtained was mixed using magnetic stirrer/vortex shaker until clear. The solution thus obtained was filtered through 0.22 μm filters.

### Step-2: Preparation of Liposomal Composition

0.5 ml of the Concentrate or Proliposomal Composition of Docetaxel, as obtained in Step-1 was rapidly injected at a rate of 0.16 ml/second using a 1 ml syringe with a hypodermic needle of gauge 30 G into 7.5 ml of 5% Dextrose solution to obtain a dispersion containing Docetaxel loaded liposomes, providing the object Liposomal Composition of Docetaxel at a drug concentration of 0.75 mg/ml.

The liposomal composition thus prepared had a particle size of approximately 85 nm and a stability of more than 12 hours.

### EXAMPLE 9

### Liposomal Composition of Docetaxel

### Step-1: Preparation of Concentrate or Proliposomal Composition

37.5 mg of Hydrogenated Soya phosphatidyl choline (HSPC, 43.61 mole %), 11.25 mg Cholesterol (25.79 mole %), 15 mg Egg phosphatidyl glycerol (EPG, 17.28 mole %), 7.5 mg of Carbonyl methoxy polyethylene glycol 2000-distearoyl phosphatidyl ethanolamine (2.46 mole %) and 0.5 mg of α-Tocopherol (0.975 mole %) were dissolved in 1 ml of absolute ethanol which was then heated at 70° C. for 2 minutes using water bath to obtain a clear solution of lipids. The solution was brought down to room temperature. 9 mg of amorphous Docetaxel (9.874 mole %) was then added to this solution. The Concentrate or Proliposomal Composition of Docetaxel so obtained was mixed using magnetic stirrer/vortex shaker until clear. The solution thus obtained was filtered through 0.22 μm filters.

### Step-2: Preparation of Liposomal Composition

1.0 ml of the Concentrate or Proliposomal Composition of Docetaxel, as obtained in Step-1 was rapidly injected at a rate of 0.20 ml/second using a 1 ml syringe with a hypodermic needle of gauge 24 G into 11 ml of 5% Dextrose solution to obtain a dispersion containing Docetaxel loaded liposomes, providing the object Liposomal Composition of Docetaxel at a drug concentration of 0.75 mg/ml.

The liposomal composition thus prepared had a particle size of approximately 85 nm and a stability of more than 5 hours.

### EXAMPLE-10

### Liposomal Composition of Docetaxel

### Step-1: Preparation of Concentrate or Proliposomal Composition

937.5 mg of Hydrogenated Soya phosphatidyl choline (HSPC, 45.165 mole %), 281.5 mg Cholesterol (26.71 mole %), 375 mg Egg phosphatidyl glycerol (EPG, 17.90 mole %), were dissolved in a mixture of 2.5 ml of propylene glycol and 10 ml of ethanol, which was then heated at 40° C. for 4 minutes using water bath to obtain a clear solution of lipids. The solution was brought down to room temperature. A solution of 225 mg of amorphous Docetaxel (10.225 mole %) in 12 ml of ethanol was then added to this solution and the volume was made up to 25 ml by addition of ethanol. The Concentrate or Proliposomal Composition of Docetaxel so obtained was mixed using magnetic stirrer/vortex shaker until clear. The solution thus obtained was filtered through 0.22 μm filters.

### Step-2: Preparation of Liposomal Composition

2.0 ml of the Concentrate or Proliposomal Composition of Docetaxel, as obtained in Step-1 was rapidly injected at a rate of 0.40 ml/second using a 2 ml syringe with a hypodermic needle of gauge 20 G into 22 ml of 5% Dextrose solution to obtain a dispersion containing Docetaxel loaded liposomes, providing the object Liposomal Composition of Docetaxel at a drug concentration of 0.75 mg/ml.

The liposomal composition thus prepared had a particle size of approximately 95 nm and a stability of more than 6 hours.

### EXAMPLE-11

### Liposomal Composition of Docetaxel

### Step-1: Preparation of Concentrate or Proliposomal Composition

937.5 mg of Hydrogenated Soya phosphatidyl choline (HSPC, 45.165 mole %), 281.5 mg Cholesterol (26.71 mole %), 375 mg Egg phosphatidyl glycerol (EPG, 17.90 mole %), were dissolved in a mixture of 2.5 ml of propylene glycol and 10 ml of ethanol, which was then heated at 40° C. for 4 minutes using water bath to obtain a clear solution of lipids. The solution was brought down to room temperature. A solution of 225 mg of amorphous Docetaxel (10.225 mole %) in 12 ml of ethanol was then added to this solution and the volume was made up to 25 ml by addition of ethanol. The Concentrate or Proliposomal Composition of Docetaxel so obtained was mixed using magnetic stirrer/vortex shaker until clear. The solution thus obtained was filtered through 0.22 μm filters.

### Step-2: Preparation of Liposomal Composition

22.7 ml of the Concentrate or Proliposomal Composition of Docetaxel, as obtained in Step-1 was rapidly injected twice at a rate of 0.6 ml/second using a 10 ml syringe with a hypodermic needle of gauge 24 G into 250 ml of 5% Dextrose solution to obtain a dispersion containing Docetaxel loaded liposomes, providing the object Liposomal Composition of Docetaxel at a drug concentration of 0.75 mg/ml.

The liposomal composition thus prepared had a particle size of approximately 98 nm and a stability of more than 6 hours.

### EXAMPLE-12

### Liposomal Composition of Docetaxel

### Step-1: Preparation of Concentrate or Proliposomal Composition

1.875 gm of Hydrogenated Soya phosphatidyl choline (HSPC, 45.165 mole %), 563 mg Cholesterol (26.71 mole %), 750 mg Egg phosphatidyl glycerol (EPG, 17.90 mole %), were dissolved in a mixture of 5 ml of propylene glycol and 20 ml of ethanol, which was then heated at 40° C. for 10 minutes using water bath to obtain a clear solution of lipids. The solution was brought down to room temperature. A solution of 450 mg of amorphous Docetaxel (10.225 mole %) in 25 ml of ethanol was then added to this solution and the volume was made up to 50 ml by addition of ethanol. The Concentrate or Proliposomal Composition of Docetaxel so obtained was mixed using magnetic stirrer/vortex shaker until clear. The solution thus obtained was filtered through 0.22 μm filters.

### Step-2: Preparation of Liposomal Composition

45.4 ml of the Concentrate or Proliposomal Composition of Docetaxel, as obtained in Step-1 was rapidly injected thrice at a rate of 0.50 ml/second using a 20 ml syringe with a hypodermic needle of gauge 21 G into 500 ml of 5% Dextrose solution to obtain a dispersion containing Docetaxel loaded liposomes, providing the object Liposomal Composition of Docetaxel at a drug concentration of 0.75 mg/ml.

The liposomal composition thus prepared had a particle size of approximately 90 nm and a stability of more than 5 hours.

### EXAMPLE 13

### Liposomal Composition of Docetaxel

### Step-1: Preparation of Concentrate or Proliposomal Composition

112.5 mg of Hydrogenated Soya phosphatidyl choline (HSPC, 45.48 mole %), 33.75 mg Cholesterol (26.89 mole %), 45 mg Egg phosphatidyl glycerol (EPG, 17.98 mole %) were dissolved in a mixture of ethanol and propylene glycol (3 ml, 9:1 ratio) which was then heated at 70° C. for 3 minutes using water bath to obtain a clear solution of lipids. The solution was brought down to room temperature. 27 mg of Docetaxel trihydrate (9.65 mole %) was then added to this solution. The Concentrate or Proliposomal Composition of Docetaxel so obtained was mixed using magnetic stirrer/vortex shaker until clear. The solution thus obtained was filtered through 0.22 μm filters.

### Step-2: Preparation of Liposomal Composition

2.0 ml of the Concentrate or Proliposomal Composition of Docetaxel, as obtained in Step-1 was rapidly injected at a rate of 0.40 ml/second using a 2 ml syringe with a hypodermic needle of gauge 26 G into 22 ml of 5% Dextrose solution to obtain a dispersion containing Docetaxel loaded liposomes, providing the object Liposomal Composition of Docetaxel at a drug concentration of 0.75 mg/ml.

The liposomal composition thus prepared had a particle size of approximately 85 nm and a stability of more than 6 hours.

### EXAMPLE 14

### Liposomal Composition of Paclitaxel

### Step-1: Preparation of Concentrate or Proliposomal Composition

37.5 mg of Hydrogenated Soya phosphatidyl choline (HSPC, 45.74 mole %), 11.25 mg Cholesterol (27.05 mole %) and 15 mg Distearoyl phosphatidyl glycerol (DSPG, 17.41 mole %) were dissolved in 1 ml of a mixture of absolute ethanol and Propylene glycol (9:1) which was then heated at 70° C. for 2 minutes using water bath to obtain a clear solution of lipids. The solution was brought down to room temperature. 9 mg Paclitaxel (9.80 mole %) was then added to this solution. The Concentrate or Proliposomal Composition of Paclitaxel so obtained was mixed using magnetic stirrer/vortex shaker until clear. The solution thus obtained was filtered through 0.22 μm filters.

### Step-2: Preparation of Liposomal Composition

1.0 ml of the Concentrate or Proliposomal Composition of Paclitaxel, as obtained in Step-1 was rapidly injected at a rate of 0.20 ml/second using a 1 ml syringe with a hypodermic needle of gauge 30 G into 11 ml of 5% Dextrose solution to obtain a dispersion containing Paclitaxel loaded liposomes, providing the object Liposomal Composition of Paclitaxel at a drug concentration of 0.75 mg/ml.

The liposomal composition thus prepared had a particle size of approximately 90 nm and a stability of more than 6 hours.

### EXAMPLE 15

### Liposomal Composition of Etoposide

### Step-1: Preparation of Concentrate or Proliposomal Composition

37.5 mg of Hydrogenated Soya phosphatidyl choline (HSPC, 43.53 mole %), 11.25 mg Cholesterol (25.74 mole %) and 15 mg Egg phosphatidyl glycerol (EPG, 17.21 mole %) were dissolved in 1 ml of absolute ethanol which was then heated at 70° C. for 2 minutes using water bath to obtain a clear solution of lipids. The solution was brought down to room temperature. 9 mg Etoposide (13.53 mole %) was then added to this solution. The Concentrate or Proliposomal Composition of Etoposide so obtained was mixed using magnetic stirrer/vortex shaker until clear. The solution thus obtained was filtered through 0.22 μm filters.

### Step-2: Preparation of Liposomal Composition

1.0 ml of the Concentrate or Proliposomal Composition of Etoposide, as obtained in Step-1 was rapidly injected at a rate of 0.40 ml/second using a 1 ml syringe with a hypodermic needle of gauge 26 G into 11 ml of 5% Dextrose solution to obtain a dispersion containing Etoposide loaded liposomes, providing the object Liposomal Composition of Etoposide at a drug concentration of 0.75 mg/ml.

The liposomal composition thus prepared had a particle size of approximately 90 nm and a stability of more than 6 hours.

### EXAMPLE 16

### Liposomal Composition of Cyclosporine A

### Step-1: Preparation of Concentrate or Proliposomal Composition

37.5 mg of Hydrogenated Soya phosphatidyl choline (HSPC, 46.76 mole %), 11.25 mg Cholesterol (27.65 mole %) and 15 mg Egg phosphatidyl glycerol (EPG, 18.49 mole %) were dissolved in 1 ml of a mixture of absolute ethanol and Propylene glycol (9:1) which was then heated at 70° C. for 2 minutes using water bath to obtain a clear solution of lipids. The solution was brought down to room temperature. 9 mg Cyclosporine A (7.11 mole %) was then added to this solution. The Concentrate or Proliposomal Composition of Cyclosporine A so obtained was mixed using magnetic stirrer/vortex shaker until clear. The solution thus obtained was filtered through 0.22 μm filters.

### Step-2: Preparation of Liposomal Composition

1.0 ml of the Concentrate or Proliposomal Composition of Cyclosporine A, as obtained in Step-1 was rapidly injected at a rate of 0.20 ml/second using a 1 ml syringe with a hypodermic needle of gauge 30 G into 11 ml of 5% Dextrose solution to obtain a dispersion containing Cyclosporine A loaded liposomes, providing the object Liposomal Composition of Cyclosporine A at a drug concentration of 0.75 mg/ml.

The liposomal composition thus prepared had a particle size of approximately 90 nm and a stability of more than 24 hours.

### EXAMPLE 17

### Liposomal Composition of Cyclosporine A

### Step-1: Preparation of Concentrate or Proliposomal Composition

37.5 mg of Hydrogenated Soya phosphatidyl choline (HSPC, 46.76 mole %), 11.25 mg Cholesterol (27.65 mole %) and 15 mg Egg phosphatidyl glycerol (EPG, 18.49 mole %) were dissolved in 1 ml of a mixture of absolute ethanol and Propylene glycol (9:1) which was then heated at 70° C. for 2 minutes using water bath to obtain a clear solution of lipids. The solution was brought down to room temperature. 9 mg Cyclosporine A (7.11 mole %) was then added to this solution. The Concentrate or Proliposomal Composition of Cyclosporine A so obtained was mixed using magnetic stirrer/vortex shaker until clear. The solution thus obtained was filtered through 0.22 μm filters.

### Step-2: Preparation of Liposomal Composition

1.0 ml of the Concentrate or Proliposomal Composition of Cyclosporine A, as obtained in Step-1 was rapidly injected at a rate of 0.14 ml/second using a 1 ml syringe with a hypodermic needle of gauge 30 G into 11 ml of 5% Dextrose solution to obtain a dispersion containing Cyclosporine A loaded liposomes, providing the object Liposomal Composition of Cyclosporine A at a drug concentration of 0.75 mg/ml.

The liposomal composition thus prepared had a particle size of approximately 90 nm and a stability of more than 10 hours.

### EXAMPLE 18

### Liposomal Composition of Betulinic Acid Derivative, DRF-4015 (III)

### Step-1: Preparation of Concentrate or Proliposomal Composition

37.5 mg of Hydrogenated Soya phosphatidyl choline (HSPC, 43.49 mole %), 11.25 mg Cholesterol (25.71 mole %) and 15 mg Egg phosphatidyl glycerol (EPG, 17.19 mole %) were dissolved in 1 ml of a mixture of absolute ethanol and Propylene glycol (9:1) which was then heated at 70° C. for 2 minutes using water bath to obtain a clear solution of lipids. The solution was brought down to room temperature. 9 mg DRF-4015 (13.60 mole %) was then added to this solution. The Concentrate or Proliposomal Composition of DRF-4015so obtained was mixed using magnetic stirrer/vortex shaker until clear. The solution thus obtained was filtered through 0.22 μm filters.

### Step-2: Preparation of Liposomal Composition

1.0 ml of the Concentrate or Proliposomal Composition of DRF-4015, as obtained in Step-1 was rapidly injected at a rate of 0.33 ml/second using a 1 ml syringe with a hypodermic needle of gauge 30 G into 11 ml of 5% Dextrose solution to obtain a dispersion containing DRF-4015 loaded liposomes, providing the object Liposomal Composition of DRF-4015 (III) at a drug concentration of 0.75 mg/ml.

The liposomal composition thus prepared had a particle size of approximately 95 nm and a stability of more than 6 hours.

### EXAMPLE 19

### Liposomal Composition of Betulinic Acid Derivative, DRF-4012 (II)

### Step-1: Preparation of Concentrate or Proliposomal Composition

37.5 mg of Hydrogenated Soya phosphatidyl choline (HSPC, 43.25 mole %), 11.25 mg Cholesterol (25.58 mole %) and 15 mg Egg phosphatidyl glycerol (EPG, 17.10 mole %) were dissolved in 1 ml of a mixture of absolute ethanol and Propylene glycol (9:1) which was then heated at 70° C. for 2 minutes using water bath to obtain a clear solution of lipids. The solution was brought down to room temperature. 9 mg DRF-4012 (14.07 mole %) was then added to this solution. The Concentrate or Proliposomal Composition of DRF-4012 so obtained was mixed using magnetic stirrer/vortex shaker until clear. The solution thus obtained was filtered through 0.22 μm filters.

### Step-2: Preparation of Liposomal Composition

1.0 ml of the Concentrate or Proliposomal Composition of DRF-4012, as obtained in Step-1 was rapidly injected at a rate of 0.50 ml/second using a 1 ml syringe with a hypodermic needle of gauge 30 G into 11 ml of 5% Dextrose solution to obtain a dispersion containing DRF-4012 loaded liposomes, providing the object Liposomal Composition of DRF-4012 (II) at a drug concentration of 0.75 mg/ml.

The liposomal composition thus prepared had a particle size of approximately 90 nm and a stability of more than 6 hours.

### EXAMPLE 20

### Liposomal Composition of Betulinic Acid Derivative, MJ-1098 (1)

### Step-1: Preparation of Concentrate or Proliposomal Composition

37.5 mg of Hydrogenated Soya phosphatidyl choline (HSPC, 43.25 mole %), 11.25 mg Cholesterol (25.58 mole %) and 15 mg Egg phosphatidyl glycerol (EPG, 17.10 mole %) were dissolved in 1 ml of a mixture of absolute ethanol, Propylene glycol, and N,N-dimethylacetamide (8:1:1) which was then heated at 70° C. for 2 minutes using water bath to obtain a clear solution of lipids. The solution was brought down to room temperature. 9 mg MJ-1098 (14.07 mole %) was then added to this solution. The Concentrate or Proliposomal Composition of MJ-1098 so obtained was mixed using magnetic stirrer/vortex shaker until clear. The solution thus obtained was filtered through 0.22 μm filters.

### Step-2: Preparation of Liposomal Composition

1.0 ml of the Concentrate or Proliposomal Composition of MJ-1098, as obtained in Step-1 was rapidly injected at a rate of 0.50 ml/second using a 1 ml syringe with a hypodermic needle of gauge 30 G into 11 ml of 5% Dextrose solution to obtain a dispersion containing MJ-1098 loaded liposomes, providing the object Liposomal Composition of MJ-1098 at a drug concentration of 0.75 mg/ml.

The liposomal composition thus prepared had a particle size of approximately 95 nm and a stability of more than 6 hours.

### EXAMPLE 21

### Liposomal Composition of Docetaxel

### Step-1: Preparation of Concentrate or Proliposomal Composition

37.5 mg of Hydrogenated Soya phosphatidyl choline (HSPC, 45.16 mole %), 11.25 mg Cholesterol (26.71 mole %) and 15 mg Egg phosphatidyl glycerol (EPG, 17.89 mole %) were dissolved in 1 ml of absolute ethanol which was then heated at 70° C. for 2 minutes using water bath to obtain a clear solution of lipids. The solution was brought down to room temperature. 9 mg of amorphous Docetaxel (10.22 mole %) was then added to this solution. The Concentrate or Proliposomal Composition of Docetaxel so obtained was mixed using magnetic stirrer/vortex shaker until clear. The solution thus obtained was filtered through 0.22 μm filters.

### Step-2: Preparation of Liposomal Composition

1.0 ml of the Concentrate or Proliposomal Composition of Docetaxel, as obtained in Step-1 was rapidly injected at a rate of 0.20 ml/second using a 1 ml syringe with a hypodermic needle of gauge 16 G into 11 ml of 5% Dextrose solution to obtain a dispersion containing Docetaxel loaded liposomes, providing the object Liposomal Composition of Docetaxel at a drug concentration of 0.75 mg/ml.

The liposomal composition thus prepared had a particle size of approximately 200 nm and a stability of less than 3 hours.

### EXAMPLE 22

### Liposomal Composition of Docetaxel

### Step-1: Preparation of Concentrate or Proliposomal Composition

37.5 mg of Hydrogenated Soya phosphatidyl choline (HSPC, 44.71 mole %), 11.25 mg Cholesterol (26.44 mole %), 15 mg Egg phosphatidyl glycerol (EPG, 17.72 mole %), and 0.50 mg of α-Tocopheryl acetate (1.0 mole %) were dissolved in 1 ml of a mixture of absolute ethanol and propylene glycol (9:1 ratio), which was then heated at 70° C. for 2 minutes using water bath to obtain a clear solution of lipids. The solution was brought down to room temperature, to which was added 9 mg of amorphous Docetaxel (10.12 mole %). The Concentrate or Proliposomal Composition of Docetaxel so obtained was mixed using magnetic stirrer/vortex shaker until clear. The solution thus obtained was filtered through 0.22 μm filters.

### Step-2: Preparation of Liposomal Composition

1.0 ml of the Concentrate or Proliposomal Composition of Docetaxel, as obtained in Step-1 was injected at a rate of 0.05 ml/second using a 1 ml syringe with a hypodermic needle of gauge 28 G into 11 ml of 5% Dextrose solution to obtain a dispersion containing Docetaxel loaded liposomes, providing the object Liposomal Composition of Docetaxel at a drug concentration of 0.75 mg/ml.

The liposomal composition thus prepared had a particle size of approximately 195 nm and a stability of less than 2 hours.

### EXAMPLE 23

### Liposomal Composition of Docetaxel

### Step-1: Preparation of Concentrate or Proliposomal Composition

37.5 mg of Hydrogenated Soya phosphatidyl choline (HSPC, 44.71 mole %), 11.25 mg Cholesterol (26.44 mole %), 15 mg Egg phosphatidyl glycerol (EPG, 17.72 mole %), and 0.50 mg of α-Tocopheryl acetate (1.0 mole %) were dissolved in 1 ml of a mixture of absolute ethanol and propylene glycol (9:1 ratio), which was then heated at 70° C. for 2 minutes using water bath to obtain a clear solution of lipids. The solution was brought down to room temperature, to which was added 9 mg of amorphous Docetaxel (10.12 mole %). The Concentrate or Proliposomal Composition of Docetaxel so obtained was mixed using magnetic stirrer/vortex shaker until clear. The solution thus obtained was filtered through 0.22 μm filters.

### Step-2: Preparation of Liposomal Composition

1.0 ml of the Concentrate or Proliposomal Composition of Docetaxel, as obtained in Step-1 was injected at a rate of 0.05 ml/second using a 1 ml syringe with a hypodermic needle of gauge 16 G into 11 ml of 5% Dextrose solution to obtain a dispersion containing Docetaxel loaded liposomes, providing the object Liposomal Composition of Docetaxel at a drug concentration of 0.75 mg/ml.

The liposomal composition thus prepared had a particle size of approximately 270 nm and a stability of less than 0.5 hours.

1. A proliposomal composition comprising a concentrate of:
a) a membrane forming lipid comprising of one or more of a saturated phospholipid, an unsaturated phospholipid, or mixtures thereof;
b) a membrane stabilizing agent selected from a sterol compound;
c) a vehicle for the lipids selected from a water-miscible organic solvent or mixtures thereof; and
d) one or more poorly water-soluble drugs and compounds,
contained in sterile glass vials, sterile vials made of non-toxic materials, or pre-filled sterile syringes, wherein the proliposomal composition forms liposomes of the one or more water-soluble drugs and compounds upon injection into a diluting fluid.

2. The composition according to claim 1, further comprising one or more of a Polyethylene Glycol (PEG)-coupled phospholipid and pharmaceutically acceptable excipients.

3. (canceled)

4. The composition according to claim 1, wherein the one or more poorly water-soluble drugs and compounds belong to the class of anticancer agents selected from Paclitaxel, Docetaxel, Irinotecan, Topotecan, SN-38, Doxorubicin, Daunomycin, Cisplatin, Oxaliplatin, 5-Fluorouracil, Mitomycin, Methotrexate, Etoposide, Wedelolactone, Betulinic acid, a Betulinic acid derivative of formula (I); a Betulinic acid of formula (II); or a Betulinic acid of formula (III); 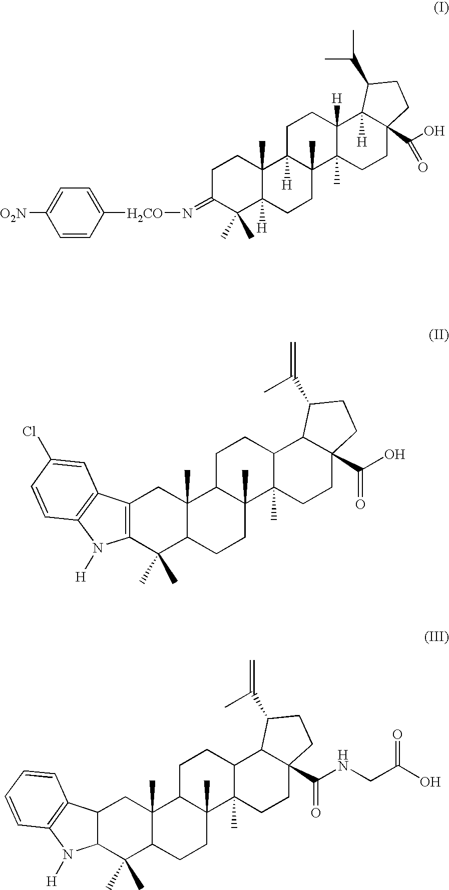
anti-inflammatory agents selected from Indomethacin, Ibuprofen, Ketoprofen, Flubiprofen, Piroxicam, Tenoxicam, or Naproxen; anti-fungal agents selected from Ketoconazole or Amphotericin B; sex hormones selected from Testosterone, Estrogen, Progesterone, or Estradiol; steroids selected from Dexamethasone, Prednisolone, Fulvestrant, Exemestane, or Triamcinolone; antihypertensive agents selected from Captopril, Ramipril, Terazosin, Minoxidil, or Parazosin; antiemetics selected from Ondansetron or Granisetron; antibiotics selected from Metronidazole or Fusidic acid; immunomodulators selected from Cyclosporine or Biphenyl dimethyl dicarboxylic acid; and anaesthetics selected from Propofol, Alfaxalone, or Hexobarbital.

5-8. (canceled)

9. A composition according to claim 1, wherein the membrane forming lipid is a saturated phospholipid selected from Hydrogenated soya phosphatidylcholine (HSPC), Hydrogenated Soya lecithin, Dimyristoyl phosphatidyl ethanolamine (DMPE), Dipalmitoyl phosphatidyl ethanolamine (DPPE), Dimyristoyl Phosphatidylcholine (DMPC), Dipalmitoyl Phosphatidylcholine (DPPC), Distearoylphosphatidyl choline (DSPC), Dilauroyl phosphatidylcholine (DLPC), 1-myristoyl-2-palmitoyl phosphatidylcholine, 1-palmitoyl-2-myristoyl phosphatidylcholine, 1-Palmitoyl phosphatidylcholine, 1-stearoyl-2-palmitoyl Phosphatidylcholine, Dipalmitoyl Sphingomyelin, Distearoyl Sphingomyelin, Hydrogenated phosphatidyl inositol (HPI), Dimyristoyl phosphatidyl glycerol (DMPG), Dipalmitoyl phosphatidyl glycerol (DPPG), Distearoyl phosphatidyl glycerol (DSPG), Dimyristoyl phosphatidic acid (DMPA), Dipalmitoyl phosphatidic acid (DPPA), Dimyristoyl phosphatidyl serine (DMPS), Dipalmitoyl phosphatidyl serine (DPPS), Diphosphatidyl glycerol (DPG), Hydrogenated Soya phosphatidyl glycerol (SPG-3), Dioleoyl phosphatidyl glycerol (DOPG), Distearoyl phosphatidic acid (DSPA), or mixtures thereof.

10-11. (canceled)

12. A composition according to claim 1, wherein the membrane forming lipid is an unsaturated phospholipid selected from Lecithin, Phosphatidylcholine (PC), Phosphatidyl ethanolamine (PE), Lysolecithin, Lysophosphatidyl ethanolamine, Dilaurylphosphatidyl choline (DLPC), Dioleoyl phosphatidyl choline (DOPC), Sphingomyelin, Brain Sphingomyelin, Cerebrosides, Egg Phosphatidyl glycerol (EPG), Soya phosphatidyl glycerol (SPG), Phosphatidyl inositol (PI), Phosphatidic acid (PA), Phosphatidyl serine (PS), Dilauroyl phosphatidyl glycerol (DLPG), Cardiolipins, or mixtures thereof.

13-14. (canceled)

15. A composition according to claim 1, wherein the membrane stabilizing agent is a sterol compound selected from Cholesterol, Cholesterol derivatives, Vitamin D, Cholesteryl esters, or mixtures thereof.

16-17. (canceled)

18. A composition according to claim 1, wherein the vehicle is a water-miscible organic solvent selected from ethanol, dimethylformamide, dimethylacetamide, dimethyl sulfoxide, diethyl sulfoxide, polyethylene glycols, and propylene glycol, or mixtures thereof.

19-20. (canceled)

21. A composition according to claim 2, wherein the Polyethylene Glycol (PEG)-coupled lipids are selected from Carbonyl methoxypolyethylene glycol-distearoyl phosphatidyl ethanolamine, Carbonyl methoxypolyethylene glycol-dipalmitoyl phosphatidyl ethanolamine, or Carbonyl methoxypolyethylene glycol-dimyristoyl phosphatidyl ethanolamine.

22-23. (canceled)

24. A composition according to claim 2, wherein the pharmaceutically acceptable excipient is an antioxidant selected from α-Tocopherol or its acetate salt, Vitamin E, β-carotene, α-Carotene, Lycopene, Lutein, or Zeaxanthine.

25-26. (canceled)

27. A composition according to claim 2, wherein pharmaceutically acceptable excipient is a buffering is selected from citrate buffer, tris-buffer, or phosphate buffer.

28-29. (canceled)

30. A liposomal comprising of:
a) a membrane forming lipid comprising of one or more of a saturated phospholipid, an unsaturated phospholipid, or mixtures thereof;
b) a membrane stabilizing agent selected from a sterol compound;
c) a vehicle for the lipids selected from a water-miscible organic solvent or mixtures thereof;
d) a diluting fluid, and
e) one or more poorly water-soluble drugs and compounds.

31. The composition according to claim 30, further comprising of a Polyethylene Glycol (PEG)-coupled phospholipid and pharmaceutically acceptable excipients.

32. A composition according to claim 30, wherein:
a. the one or more poorly water-soluble drugs and compounds have water solubility of less than 10 mg/ml;
b. the membrane forming lipids are saturated phospholipids selected from Hydrogenated soya phosphatidylcholine (HSPC), Hydrogenated Soya lecithin, Dimyristoyl phosphatidyl ethanolamine (DMPE), Dipalmitoyl phosphatidyl ethanolamine (DPPE), Dimyristoyl Phosphatidylcholine (DMPC), Dipalmitoyl Phosphatidylcholine (DPPC), Distearoylphosphatidyl choline (DSPC), Dilauroyl phosphatidylcholine (DLPC), 1-myristoyl-2-palmitoyl phosphatidylcholine, 1-palmitoyl-2-myristoyl phosphatidylcholine, 1-Palmitoyl phosphatidylcholine, 1-stearoyl-2-palmitoyl Phosphatidylcholine, Dipalmitoyl Sphingomyelin, Distearoyl Sphingomyelin, Hydrogenated phosphatidyl inositol (HPI), Dimyristoyl phosphatidyl glycerol (DMPG), Dipalmitoyl phosphatidyl glycerol (DPPG), Distearoyl phosphatidyl glycerol (DSPG), Dimyristoyl phosphatidic acid (DMPA), Dipalmitoyl phosphatidic acid (DPPA), Dimyristoyl phosphatidyl serine (DMPS), Dipalmitoyl phosphatidyl serine (DPPS), Diphosphatidyl glycerol (DPG), Hydrogenated Soya phosphatidyl glycerol (SPG-3), Dioleoyl phosphatidyl glycerol (DOPG), Distearoyl phosphatidic acid (DSPA), or mixtures thereof;
c. the membrane stabilizing agents are sterol compounds selected from Cholesterol, Cholesterol derivatives, Vitamin D, Cholesteryl esters, or mixtures thereof; and
d. the vehicles are water-miscible organic solvents selected from ethanol, dimethylformamide, dimethylacetamide, dimethyl sulfoxide, diethyl sulfoxide, polyethylene glycols propylene glycol, or mixtures thereof.

33-37. (canceled)

38. A composition according to claim 31, wherein:
a) the Polyethylene Glycol (PEG)-coupled lipids are selected from Carbonyl methoxypolyethylene glycol-distearoyl phosphatidyl ethanolamine, Carbonyl methoxypolyethylene glycol-dipalmitoyl phosphatidyl ethanolamine, or Carbonyl methoxypolyethylene glycol-dimyristoyl phosphatidyl ethanolamine; and
b) the pharmaceutically acceptable excipients are antioxidants selected from α-Tocopherol or its acetate salt, Vitamin E, β-carotene, α-Carotene, Lycopene, Lutein, or Zeaxanthine.

39. (canceled)

40. A process for preparation of the proliposomal composition comprising
a membrane forming lipid comprising of one or more of a saturated phospholipid, an unsaturated phospholipid, or mixtures thereof;
a membrane stabilizing agent selected from a sterol compound; and
a vehicle for the lipids selected from a water-miscible organic solvent or mixtures thereof; and
one or more poorly water-soluble drugs and compounds,
wherein the process comprises the steps of:
a) mixing together the membrane forming lipids and the membrane stabilizing agent in the vehicle at a temperature of between 30° C. to 70° C. to obtain a clear solution;
b) cooling the clear solution of step a) to room temperature;
c) adding one or more poorly water-soluble drugs and compounds either as a solid or as a mixture in the vehicle to the solution of step b);
d) mixing the contents of step c) to obtain a clear solution;
e) diluting the mixture of step d) with the vehicle;
f) filtering the solution of step e) through sterile filters to obtain a concentrate of the proliposomal composition; and
g) filling the concentrate of step f) into glass vials, vials made of non-toxic materials, or syringes.

41. (canceled)

42. A process for preparation of the proliposomal composition comprising
a membrane forming lipid comprising of one or more of a saturated phospholipid, an unsaturated phospholipid, or mixtures thereof;
a membrane stabilizing agent selected from a sterol compound; and
a vehicle for the lipids selected from a water-miscible organic solvent or mixtures thereof;
one or more poorly water-soluble drugs and compounds, and
one or more of a Polyethylene Glycol (PEG)-coupled phospholipid and pharmaceutically acceptable excipients, wherein the process comprises the steps of:
a) mixing together the membrane forming lipids, the membrane stabilizing agent, the (PEG)-coupled phospholipids, and the pharmaceutically acceptable antioxidant and/or the pharmaceutically acceptable acidifying agent in the vehicle, at a temperature of between 30° C. to 70° C. to obtain a clear solution;
b) cooling the clear solution of step a) to room temperature;
c) adding one or more poorly water-soluble drugs and compounds either as a solid or as a mixture in the vehicle to the solution of step b);
d) mixing the contents of step c) to obtain a clear solution;
e) optionally adjusting the pH of the solution of step d) with a pharmaceutically acceptable buffering agent;
f) diluting the mixture of step d) or e) further with the vehicle;
g) filtering the solution of step f) through sterile filters to obtain a concentrate of the proliposomal composition; and
h) filling the concentrate of step g) into glass vials, vials made of non-toxic materials, or syringes.

43. (canceled)

44. A process for preparation of the liposomal composition comprising
a) a membrane forming lipid comprising of one or more of a saturated phospholipid, an unsaturated phospholipid, or mixtures thereof;
b) a membrane stabilizing agent selected from a sterol compound;
c) a vehicle for the lipids selected from a water-miscible organic solvent or mixtures thereof;
d) a diluting fluid; and
e) one or more poorly water-soluble drugs and compounds
wherein the liposomal composition is characterized by a physical stability of not less than 4 hours, ≧95% encapsulation of the one or more poorly water-soluble drugs and compounds in the liposomes, having a particle size diameter of less than 100 nm, comprising injection of the concentrate of the proliposomal composition of claim 1, through syringes, fitted with hypodermic needles of gauge 18 G to 30 G into a diluting fluid at a rate of about 0.10 ml/second to about 1.5 ml/second.

45-46. (canceled)

47. A method of treatment of pathological conditions in humans and other animals comprising administration of a liposomal composition comprising
a membrane forming lipid comprising of one or more of a saturated phospholipid, an unsaturated phospholipid, or mixtures thereof;
b) a membrane stabilizing agent selected from a sterol compound;
c) a vehicle for the lipids selected from a water-miscible organic solvent or mixtures thereof;
d) a diluting fluid, and
e) one or more poorly water-soluble drugs and compounds.

48. The method according to claim 47, wherein the one or more poorly water-soluble drugs and compounds belong to the class of anticancer agents selected from Paclitaxel, Docetaxel, Irinotecan, Topotecan, SN-38, Doxorubicin, Daunomycin, Cisplatin, Oxaliplatin, 5-Fluorouracil, Mitomycin, Methotrexate, Etoposide, Wedelolactone, Betulinic acid, a Betulinic acid of formula (I); a Betulinic acid of formula (II); or a Betulinic acid of formula (III); 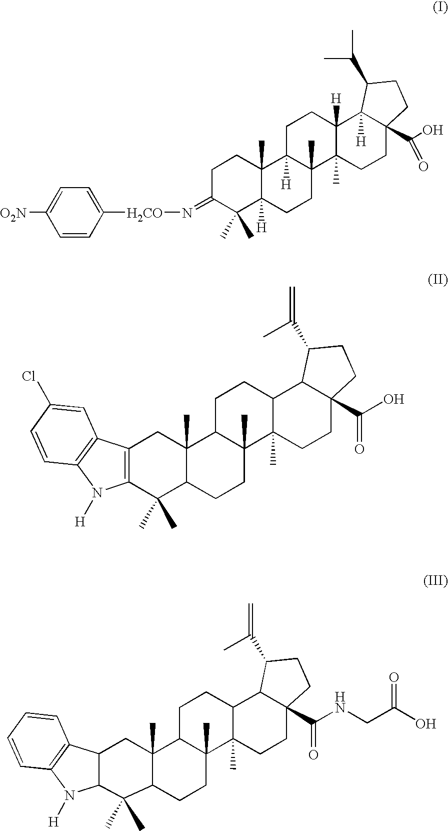
anti-inflammatory agents selected from Indomethacin, Ibuprofen, Ketoprofen, Flubiprofen, Piroxicam, Tenoxicam, or Naproxen; anti-fungal agents selected from Ketoconazole, or Amphotericin B; sex hormones selected from Testosterone, Estrogen, Progesterone, or Estradiol; steroids selected from Dexamethasone, Prednisolone, Fulvestrant, Exemestane, or Triamcinolone; antihypertensive agents selected from Captopril, Ramipril, Terazosin, Minoxidil, or Parazosin; antiemetics selected from Ondansetron or Granisetron; antibiotics selected from Metronidazole or Fusidic acid; immunomodulators selected from Cyclosporine or Biphenyl dimethyl dicarboxylic acid; and anaesthetics selected from Propofol, Alfaxalone, or Hexobarbital.

49-51. (canceled)

52. A method according to claim 47, wherein the method comprises intravenous, intramuscular, or subcutaneous injections.
